(19) United States
(12) Patent Application Publication
(10) Pub. No.: US 2024/0425568 A1
(43) Pub. Date: Dec. 26, 2024

(54) COMBINATION THERAPIES FOR HIV INFECTIONS AND USES THEREOF

(71) Applicant: VIIV HEALTHCARE COMPANY, Wilmington, DE (US)

(72) Inventors: Robert FERRIS, Durham, NC (US); Heather MADSEN, Durham, NC (US); Hangfei QI, Durham, NC (US)

(73) Assignee: VIIV HEALTHCARE COMPANY, Wilmington, DE (US)

(21) Appl. No.: 18/718,857

(22) PCT Filed: Dec. 16, 2022
(86) PCT No.: PCT/US2022/081724

§ 371 Date: Jun. 12, 2024

Related U.S. Application Data
(60) Provisional application No. 63/290,758, filed on Dec. 17, 2021.

(51) Int. Cl. C07K 16/10; A61K 31/496; A61K 31/4985; A61K 31/675; A61K 39/00; A61P 31/18

(52) U.S. Cl. CPC C07K 16/1063; A61K 31/496; A61K 31/4985; A61K 31/675; A61P 31/18; A61K 2039/505

(57) ABSTRACT

The disclosure relates to therapeutic methods or methods of treating, clearing, preventing or curing Human Immunodeficiency Virus (HIV) infection. The disclosure provides a combination of at least one agent selected from the group consisting of: fostemsavir and temsavir, or a pharmaceutically acceptable salt thereof and a CD4 binding site (CD4bs) binding protein for the use in treatment of HIV and/or clearance of HIV infected cells.

### CROSS-REFERENCE TO RELATED APPLICATIONS

This application claims the benefit of U.S. Provisional Application No. 63/290,758, filed Dec. 17, 2021, the disclosure of which is incorporated herein in it's entirety.

### SEQUENCE LISTING

The instant application contains a Sequence Listing, which has been submitted electronically in computer readable form in XML file format and is hereby incorporated by reference in its entirety. Said XML file, created on Dec. 15, 2022, is named “70090WO01_SeqList_Updated_Dec. 5, 2022” and is 15,605 bytes in size.

### FIELD OF THE INVENTION

The present invention relates to methods of treating, preventing or curing Human Immunodeficiency Virus (HIV) infection. In particular, the invention relates to methods of treatment, clearance, prevention, or cure of HIV infected cells with combinations of drugs. In some embodiments, the combination comprises an agent comprising at least one agent selected from the group consisting of: fostemsavir and temsavir, or a pharmaceutically acceptable salt thereof, and an agent comprising a CD4 binding site binding protein or a broadly neutralizing antibody in therapeutic or prophylactic applications to treat, clear, prevent, and/or cure HIV infection.

### BACKGROUND OF THE INVENTION

Human Immunodeficiency Virus (HIV), the virus that causes Acquired Immunodeficiency Syndrome (AIDS), is one of the world's most serious public health challenges. It remains a major medical problem and since the beginning of the epidemic, 79.3 million people have been infected with HIV and 36.3 million people have died of HIV. Globally, 37.7 million people were living with HIV at the end of 2020. An estimated 0.7% of adults aged 15-49 years worldwide are living with HIV, although the burden of the epidemic continues to vary considerably between countries and regions.

HIV, a retrovirus, replicates by assembling new virus particles (virions) inside an infected host cell. These new virions leave the infected host cell, now known as a producer cell, and spread the infection to other susceptible host cells. Virion morphogenesis, within the producer cell, can be divided into three stages: assembly, budding and maturation. During the assembly stage, the virion packages all of the components required for infectivity, including the gp160 viral envelope (ENV) protein. The ENV is an integral membrane protein for the fusion of the virion to target cells. As part of the packaging process, the ENV protein is inserted, co-translationally, into endoplasmic reticulum (ER) membranes of infected producer cells and then travels through the cellular secretory pathway where it is glycosylated, assembled into trimeric complexes, processed into the transmembrane (gp41) and surface (gp120) subunits by the cellular protease furin and delivered to the plasma membrane via vesicular transport. Sundquist, W. I., & Kräusslich, H. G. (2012). HIV-1 assembly, budding, and maturation. Cold Spring Harbor perspectives in medicine, 2 (7), a006924. Thus, as the process of assembly proceeds, an infected host cell presents HIV characteristic ENV on its cell surface.

The ENV presented on the infected cell surface can be an attractive target for clearance upon antibody recognition, which may result in antibody mediated infected cell death. However, the ENV on the surface of infected cells may present multiple conformations governed by interactions with cis-expressed host CD4 receptors, thereby reducing antibody recognition. As such, there is a need to increase antibody recognition of infected host cell expressed ENV.

### SUMMARY OF THE INVENTION

In one aspect, there is provided a method of treating or preventing Human HIV comprising administering a therapeutically effective amount of two or more agents selected from the group consisting of: a CD4bs binding protein, a gp120 binding protein, a broadly neutralizing antibody or an antigen binding fragment thereof, fostemsavir or a pharmaceutically acceptable salt thereof, temsavir or a pharmaceutically acceptable salt thereof, and an integrase inhibitor or a pharmaceutically acceptable salt thereof.

In one aspect, there is provided a method for the treatment or prevention of HIV infections in a human in need thereof comprising administering a therapeutically effective amount of: (a) a first agent that comprises at least one agent selected from the group consisting of: fostemsavir and temsavir, or a pharmaceutically acceptable salt thereof; and (b) a second agent that comprises at least one broadly neutralizing antibody or an antigen binding fragment thereof.

In one aspect, there is provided a method for the treatment or prevention of HIV infections in a human in need thereof comprising administering a therapeutically effective amount of: (a) a first agent that comprises at least one agent selected from the group consisting of: fostemsavir and temsavir, or a pharmaceutically acceptable salt thereof; (b) a second agent that comprises at least one broadly neutralizing antibody or an antigen binding fragment thereof; and (c) a third agent comprising at least one integrase inhibitor or a pharmaceutically acceptable salt thereof.

In one aspect, there is provided a combination for use in the treatment or prevention of HIV, comprising administering to a human a first pharmaceutical composition comprising the first agent that comprises at least one agent selected from the group consisting of: fostemsavir and temsavir, or a pharmaceutically acceptable salt thereof and a second pharmaceutical composition comprising the second agent that comprises at least one broadly neutralizing antibody or an antigen binding fragment thereof. In some embodiments, the combination for use further comprises administering a third pharmaceutical composition comprising the third agent comprising at least one integrase inhibitor or a pharmaceutically acceptable salt thereof.

In one aspect, there is provided a kit comprising the first pharmaceutical composition comprising the first agent that comprises at least one agent selected from the group consisting of: fostemsavir and temsavir, or a pharmaceutically acceptable salt thereof, the second pharmaceutical composition comprising the second agent that comprises at least one broadly neutralizing antibody or an antigen binding fragment thereof, and optionally the third pharmaceutical composition comprising the third agent comprising at least one integrase inhibitor or a pharmaceutically acceptable salt thereof.

### DESCRIPTION OF DRAWINGS/FIGURES

FIG. 1: Temsavir enhancement of the mean fluorescent intensity (MFI) of bnAb binding to HIV infected cells.

FIG. 2: Temsavir enhancement of percentage of bnAb bound HIV infected cells.

FIG. 3: Dose dependent temsavir enhancement of antibody-dependent effector cell mediated cytotoxicity (ADCC) against HIV infected primary cells.

FIG. 4: Temsavir does not lead to cell death against HIV infected primary cells in the absence of effector cells.

### DETAILED DESCRIPTION OF THE INVENTION

### Definitions

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below.

All publications, patent applications, patents, and other references mentioned herein are incorporated by reference in their entirety.

In addition, the materials, methods, and examples are illustrative only and not intended to be limiting. The details of one or more embodiments of the invention are set forth in the accompanying description below. Other features, objects, and advantages of the invention will be apparent from the description and from the claims.

### HIV

Human Immunodeficiency Virus (HIV) which unless further clarified is intended to mean HIV-1. The invention may also be effective against HIV-2, or against patients having dual HIV-1/HIV-2 infection. Human Immunodeficiency Virus type 1 (HIV-1) infection, and the resulting acquired immunodeficiency syndrome (AIDS), remain threats to global public health, despite extensive efforts to develop anti-HIV-1 therapeutic agents. An enveloped virus, HIV-1 hides from humoral recognition behind a wide array of protective mechanisms. The major HIV-1 envelope protein (HIV-1 Env) is a glycoprotein of approximately 160 kD (gp160). During infection, proteases of the host cell cleave gp160 into gp120 and gp41. gp41 is an integral membrane protein, while gp120 protrudes from the mature virus. gp120 binds to the host cell receptor, CD4, through a CD4 binding site. Together gp120 and gp41 make up the HIV-1 envelope spike, which is a target for neutralizing antibodies. Broadly neutralizing antibodies that bind to HIV-1 Env have been identified, including the N6LS antibody, which specifically binds to the CD4-binding site of gp120 and can neutralize a high percentage of HIV-1 strains.

### Binding Protein

The term “CD4 binding site (CD4bs) binding protein” as used herein refers to antibodies and other protein constructs, such as domains, that are capable of binding to the CD4 binding site of the HIV envelope glycoprotein, gp120. The terms “CD4bs binding protein,” “CD4bs binding domain” and “antigen binding protein” are used interchangeably herein. This does not include the natural cognate ligand or receptor. In some embodiments, monoclonal antibodies and antigen binding fragments thereof that bind to the CD4 binding site on gp120 and neutralize HIV-1 are provided herein.

### Antibody

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)2, Fv, disulfide linked Fv, single chain Fv, disulfide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

The term “full antibody or immunoglobulin,” “whole antibody or immunoglobulin,” or “intact antibody or immunoglobulin,” used interchangeably herein, refers to a heterotetrameric glycoprotein with an approximate molecular weight of 150,000 Daltons. An intact antibody is composed of two identical heavy chains (HCs) and two identical light chains (LCs) linked by covalent disulfide bonds. This H2L2 structure folds to form three functional domains comprising two antigen-binding fragments, known as ‘Fab’ fragments, and a ‘Fc’ crystallizable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or A light chain. The majority of antibodies in the serum belong to the IgG class, there are four isotypes of human IgG (IgG1, IgG2, IgG3 and IgG4), the sequences of which differ mainly in their hinge region.

Fully human antibodies can be obtained using a variety of methods, for example using yeast-based libraries or transgenic animals (e.g., mice) that are capable of producing repertoires of human antibodies. Yeast presenting human antibodies on their surface that bind to an antigen of interest can be selected using FACS (Fluorescence-Activated Cell Sorting) based methods or by capture on beads using labeled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an Avimer (see, e.g., U.S. Patent Application Publication Nos. 2005/0053973, 2005/0089932, 2005/0164301) or an EGF domain.

The term “broadly neutralizing antibody” (bnAb) is defined as an antibody which inhibits viral attachment and cell entry via binding to the HIV envelope glycoprotein (Env) (e.g., gp160, gp120, gp41), as a non-limiting example, by a 50% inhibition of infection in vitro, in more than 50%, 60%, 70%, 80%, 90%, 95%, 99% or greater, of a large panel of (greater than 100) HIV-1 envelope pseudotyped viruses and viral isolates. See e.g., US Published patent application No. 20120121597; Burton et al., Broadly Neutralizing Antibodies to HIV and Their Role in Vaccine Design. Annu Rev Immunol. 2016 May 20; 34:635-59.

For example, the broadly neutralizing antibody can include, but is not limited to, 2G12, 2F5, 3BC176, 3BNC60, 3BNC1 17, 4E10, 8ANC131, 8ANC195, 10E8, 10-1074, 12A12, 35022, b12, B2530, CH01-04, CH103, CH31, HJ16, M66.6, N6, N6LS, NIH45-46, PG9, PG16, PGDM1400, PGT121, PGT128, PGT135, PGT141-PGT145, PGT151, PGV04, VRC01, VRC01-LS, VRC07, VRC07-523, VRC07-LS, Z13, or any other broadly neutralizing antibodies disclosed herein. In some embodiments, the broadly neutralizing antibody binds to the HIV envelope glycoprotein. In some embodiments, the broadly neutralizing antibody binds to the HIV envelope glycoprotein selected from the group consisting of: gp160, gp120, and gp41. In one embodiment, the broadly neutralizing antibody binds to the HIV envelope glycoprotein gp41. In some embodiments, the broadly neutralizing antibody binds to gp120. In some embodiments, the broadly neutralizing antibody binds to gp120 and neutralize HIV-1. An example of VRC07-523 is set forth in J. Virol, 88 (21): pp. 12669-12682 (November 2014). An example of 3BNC1 17 is set forth in U.S. Publication No. 20140212458. An example of NIH45-46 is set forth in U.S. Publication No. 20150274813. An example of PGV04 is set forth in U.S. Publication No. 20130251726. An example of b12 is set forth in U.S. Publication No. 20160009789. An example of CH31 is set forth in U.S. Publication No. 20130251726. An example of CH103 is set forth in U.S. Publication No. 20140212458. In another embodiment, there is provided an antibody that binds HIV envelope glycoprotein at the gp120-gp41 interface. Such antibodies including, without limitation, an antibody selected from 8ANC195, 35022, and PGT151. An example of 8ANC195 is set forth in U.S. Publication No. 20150361160. An example of 35022 is set forth in U.S. Publication No. 20160022803. An example of PGT151 is set forth in U.S. Publication No. 20150152167. In another embodiment, there is provided an antibody that binds to the gp41 membrane-proximal external region (MPER) including, without limitation, 4E10, 10E8, 2F5 and Z13e1. An example of 4E10 is set forth in U.S. Publication No. 20160009789. An example of 10E8 is set forth in PCT Published Application No. WO2013070776. An example of 2F5 is set forth in U.S. Publication No. 20150158934. An example of Z13e1 is set forth in U.S. Publication No. 20120269821.

In some embodiments, the broadly neutralizing antibody is selected from the group consisting of VRC01, VRC01-LS, N6, N6LS, N6-DE, N6-LAGA, VRC07 and VRC07-523. An example of a disclosure of VRC01 is set forth in U.S. Pat. No. 8,637,036. An example of a disclosure of VRC01-LS is set forth in WO 2012/106578. Examples of disclosures of N6 and N6LS are set forth in WO 2016/196975. Examples of disclosures of VRC07 and VRC07-523 are set forth in U.S. Pat. No. 8,637,036, US Patent Publication No. 2014/0322163 A1, WO 2016/196975 and WO 2017/79479.

### Antigen Binding Site

Antigen binding site refers to a site on an antigen binding protein that is capable of specifically binding to an antigen, this may be a single variable domain, or it may be paired VH/VL domains as can be found on a standard antibody. Single-chain Fv (ScFv) domains can also provide antigen-binding sites.

### CDR

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example, those set out in Chothia et al. (1989) Nature 342:877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/ | 26-32/33/34 | 26-35/ | 30-35/ |
| | 35B | | 35A/35B | 35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

### CDR Variant

CDRs may be modified by at least one amino acid substitution, deletion or addition, wherein the variant antigen binding protein substantially retains the biological characteristics of the unmodified protein.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example, 1 or 2 amino acids, for example, 1 amino acid. Typically, the modification is a substitution, particularly a conservative substitution, for example as shown in Table 2 below.

TABLE 2

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence | Gly, Pro |
| chain orientation | |
| Aromatic | Trp, Tyr, Phe |

For example, in a variant CDR, the flanking residues that comprise the CDR as part of the alternative definition(s) e.g., Kabat or Chothia, may be substituted with a conservative amino acid residue.

Such antigen binding proteins comprising variant CDRs as described above may be referred to herein as “functional CDR variants”.

### Epitope

The term “epitope” as used herein refers to that portion of the antigen that makes contact with a particular binding domain of the antigen binding protein, also known as the paratope. An epitope may be linear or conformational/discontinuous. A conformational or discontinuous epitope comprises amino acid residues that are separated by other sequences, i.e. not in a continuous sequence in the antigen's primary sequence assembled by tertiary folding of the polypeptide chain. Although the residues may be from different regions of the polypeptide chain, they are in close proximity in the three dimensional structure of the antigen. In the case of multimeric antigens, a conformational or discontinuous epitope may include residues from different peptide chains. Particular residues comprised within an epitope can be determined through computer modeling programs or via three-dimensional structures obtained through methods known in the art, such as X-ray crystallography. Epitope mapping can be carried out using various techniques known to persons skilled in the art as described in publications such as Methods in Molecular Biology ‘Epitope Mapping Protocols’, Mike Schutkowski and Ulrich Reineke (volume 524, 2009) and Johan Rockberg and Johan Nilvebrant (volume 1785, 2018). Exemplary methods include peptide based approaches such as pepscan whereby a series of overlapping peptides are screened for binding using techniques such as ELISA or by in vitro display of large libraries of peptides or protein mutants, e.g. on phage. Detailed epitope information can be determined by structural techniques including X-ray crystallography, solution nuclear magnetic resonance (NMR) spectroscopy and cryogenic-electron microscopy (cryo-EM). Mutagenesis, such as alanine scanning, is an effective approach whereby loss of binding analysis is used for epitope mapping. Another method is hydrogen/deuterium exchange (HDX) combined with proteolysis and liquid-chromatography mass spectrometry (LC-MS) analysis to characterize discontinuous or conformational epitopes.

### Percent Identity

“Percent identity” or “% identity” between a query nucleic acid sequence and a subject nucleic acid sequence is the “Identities” value, expressed as a percentage, that is calculated using a suitable algorithm (e.g. BLASTN, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query nucleic acid sequence may be described by a nucleic acid sequence disclosed herein, in particular in one or more of the claims.

The query sequence may be 100% identical to the subject sequence, or it may include up to a certain integer number of amino acid or nucleotide alterations as compared to the subject sequence such that the % identity is less than 100%. For example, the query sequence is at least 50%, 60%, 70%, 75%, 80%, 85%, 90%, 95%, 96%, 97%, 98%, or 99% identical to the subject sequence. In the case of nucleic acid sequences, such alterations include at least one nucleotide residue deletion, substitution or insertion, wherein said alterations may occur at the 5′- or 3′-terminal positions of the query sequence or anywhere between those terminal positions, interspersed either individually among the nucleotide residues in the query sequence or in one or more contiguous groups within the query sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

For antibody sequences, the % identity may be determined across the entire length of the query sequence, including the CDRs. Alternatively, the % identity may exclude one or more or all of the CDRs, for example, all of the CDRs are 100% identical to the subject sequence and the % identity variation is in the remaining portion of the query sequence, e.g., the framework sequence, so that the CDR sequences are fixed and intact. The variant sequence substantially retains the biological characteristics of the unmodified protein.

### Sequence Variation

The VH or VL (or HC or LC) sequence may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s).

The HC sequence may be a variant sequence with up to 20% sequence variation.

The LC sequence may be a variant sequence with up to 20% sequence variation.

The sequence variation may exclude one or more or all of the CDRs, for example, the CDRs are the same as the VH or VL (or HC or LC) sequence and the variation is in the remaining portion of the VH or VL (or HC or LC) sequence, so that the CDR sequences are fixed and intact.

Typically, the variation is a substitution, particularly a conservative substitution, for example as shown in Table 2. The variant sequence substantially retains the biological characteristics of the unmodified protein, such as N6.

### Fc Modifications

Fc engineering methods can be applied to modify the functional or pharmacokinetic properties of an antibody. Effector function may be altered by making mutations in the Fc region that increase or decrease binding to C1q or Fcγ receptors and modify antibody-mediated complement activation including complement-dependent cytotoxicity (CDC) or antibody-dependent cell-mediated cytotoxicity (ADCC) activity respectively. Modifications to the glycosylation pattern of an antibody can also be made to change the effector function. The in vivo half-life of an antibody can be altered by making mutations that affect binding of the Fc to the FcRn (Neonatal Fc Receptor).

### Effector Function

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antigen binding protein of the present invention can be assessed for ADCC effector function in a Natural Killer cell assay. Examples of such assays can be found in Shields et al., 2001, The Journal of Biological Chemistry, Vol. 276, p. 6591-6604; Chappel et al., 1993, The Journal of Biological Chemistry, Vol 268, p. 25124-25131; Lazar et al., 2006, PNAS, 103; 4005-4010.

Examples of assays to determine CDC function include those described in J Imm Meth, 1995, 184:29-38. The effects of mutations on effector functions (e.g., FcRn binding, FcγRs and C1q binding, CDC, ADCML, ADCC, ADCP) can be assessed, e.g., as described in Grevys et al., J Immunol. 2015 Jun. 1; 194 (11): 5497-5508, or Tam et al., Antibodies 2017, 6 (3); Monnet et al., 2014 mAbs, 6:2, 422-436.

Throughout this specification, amino acid residues in Fc regions, in antibody sequences or full-length antigen binding protein sequences, are numbered according to the EU index numbering convention.

### Enhanced Effector Function

Human IgG1 constant regions containing specific mutations have been shown to enhance binding to Fc receptors. In some cases, these mutations have also been shown to enhance effector functions, such as ADCC and CDC, as described below. Antigen binding proteins of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science 2014; 343:1260-1293) and E345R/E430G/S440Y results in preformed IgG hexamers (Wang et al., Protein Cell. 2018 January; 9 (1): 63-73).

Enhanced ADCC: Fc engineering can be used to enhance ADCC. For example (with reference to IgG1), F243L/R292P/Y300L/V305I/P396L; S239D/1332E; and S298A/E333A/K334A increase FcγRIIIa binding; S239D/1332E/A330L increases FcγRIIIa binding and decreases FcγRIIb binding; G236A/S239D/1332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., 2013).

Enhanced ADCP: Fc engineering can be used to enhance ADCP. For example (with reference to IgG1), G236A/S239D/1332E increases FcγRIIa binding and increases FcγRIIIa binding (Richards J et al., Mol. Cancer Ther. 2008; 7:2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding (Wang et al. 2018).

### Decreased Effector Function

Some isotypes of human constant regions, in particular, IgG4 and IgG2 isotypes, essentially lack the functions of a) activation of complement by the classical pathway; and b) ADCC. Various modifications to the heavy chain constant region of antigen binding proteins may be carried out to alter effector function depending on the desired effector property. IgG1 constant regions containing specific mutations that reduce binding to Fc receptors and reduce an effector function, such as ADCC and CDC, have been described (Duncan et al. Nature 1988, 332; 563-564; Lund et al. J. Immunol. 1991, 147; 2657-2662; Chappel et al. PNAS 1991, 88; 9036-9040; Burton and Woof, Adv. Immunol. 1992, 51; 1-84; Morgan et al., Immunology 1995, 86; 319-324; Hezareh et al., J. Virol. 2001, 75 (24); 12161-12168).

In one embodiment of the present invention, there is provided an antigen binding protein comprising a constant region such that the antigen binding protein has reduced effector function, such as reduced ADCC and/or CDC. In one such embodiment, the heavy chain constant region may comprise a naturally disabled constant region of an IgG2 or IgG4 isotype or a mutated IgG1 constant region. Examples of suitable modifications are described in EP0307434. One example comprises substitution with alanine at positions 235 and 237 (EU index numbering), i.e., L235A and G237A (commonly referred to as “LAGA” mutations). Another example comprises substitution with alanine at positions 234 and 235 (EU index numbering), i.e., L234A and L235A (commonly referred to as “LALA” mutations). Further examples, described in EP2691417 and U.S. Pat. No. 8,969,526, comprise P329G or P329R, in combination with the LALA mutations (EU index numbering) for IgG1 Fcs and P329G or P329R in combination with S228P and L235E for IgG4 Fcs (EU index numbering).

### Mutations to Increase Half-Life by Adding or Increasing FcRn Binding

“Half-life” refers to the time required for the serum concentration of an antigen binding protein to reach half of its original value. The serum half-life of proteins can be measured by pharmacokinetic studies according to the method described by Kim et al., 1994, Eur. J. of Immuno. 24:542-548. According to this method, radio-labelled protein is injected intravenously into mice and its plasma concentration is periodically measured as a function of time, for example, at about 3 minutes to about 72 hours after the injection. Other methods for pharmacokinetic analysis and determination of the half-life of a molecule will be familiar to those skilled in the art.

Antigen binding proteins of the present invention may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428L/N434S (commonly referred to as “LS” mutations) increase FcRn binding at pH 6.0 (Wang et al. 2018).

Half-life can also be enhanced by T250Q/M428L, V259I/V308F/M428L, N434A, and T307A/E380A/N434A mutations (with reference to IgG1 and Kabat numbering) (Monnet et al.).

Half-life and FcRn binding can also be extended by introducing H433K and N434F mutations (commonly referred to as “HN” or “NHance” mutations) (with reference to IgG1) (WO2006/130834).

WO00/42072 discloses a polypeptide comprising a variant Fc region with altered FcRn binding affinity, which polypeptide comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386,388, 400, 413, 415, 424, 433, 434, 435, 436, 439, and 447 of the Fc region (EU index numbering).

WO02/060919 discloses a modified IgG comprising an IgG constant domain comprising one or more amino acid modifications relative to a wild-type IgG constant domain, wherein the modified IgG has an increased half-life compared to the half-life of an IgG having the wild-type IgG constant domain, and wherein the one or more amino acid modifications are at one or more of positions 251, 253, 255, 285-290, 308-314, 385-389, and 428-435.

Shields et al. (2001, J Biol Chem; 276:6591-604) used alanine scanning mutagenesis to alter residues in the Fc region of a human IgG1 antibody and then assessed the binding to human FcRn. Positions that effectively abrogated binding to FcRn when changed to alanine include I253, S254, H435, and Y436. Other positions showed a less pronounced reduction in binding as follows: E233-G236, R255, K288, L309, S415, and H433. Several amino acid positions exhibited an improvement in FcRn binding when changed to alanine; notable among these are P238, T256, E272, V305, T307, Q311, D312, K317, D376, E380, E382, S424, and N434. Many other amino acid positions exhibited a slight improvement (D265, N286, V303, K360, Q362, and A378) or no change (S239, K246, K248, D249, M252, E258, T260, S267, H268, S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding.

The most pronounced effect with respect to improved FcRn binding was found for combination variants. At pH 6.0, the E380A/N434A variant showed over 8-fold better binding to FcRn, relative to native IgG1, compared with 2-fold for E380A and 3.5-fold for N434A. Adding T307A to this resulted in a 12-fold improvement in binding relative to native IgG1. In one embodiment the antigen binding protein of the invention comprises the E380A/N434A mutations and has increased binding to FcRn.

Dall'Acqua et al. (2002, J Immunol.; 169:5171-80) describes random mutagenesis and screening of human IgG1 hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur when substituting residues located in a band across the Fc-FcRn interface (M252, S254, T256, H433, N434, and Y436) and to lesser extent substitutions of residues at the periphery, such as V308, L309, Q311, G385, Q386, P387, and N389. The variant with the highest affinity to human FcRn was obtained by combining the M252Y/S254T/T256E (“YTE”) and H433K/N434F/Y436H mutations and exhibited a 57-fold increase in affinity relative to the wild-type IgG1. The in vivo behavior of such a mutated human IgG1 exhibited a nearly 4-fold increase in serum half-life in cynomolgus monkeys as compared to wild-type IgG1.

The present invention therefore provides an antigen binding protein with optimized binding to FcRn. In a preferred embodiment, the antigen binding protein comprises at least one amino acid modification in the Fc region of said antigen binding protein, wherein said modification is at an amino acid position selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region.

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713) or fusing with FcRn binding domains of antibodies (WO00/09560, U.S. Pat. No. 4,703,039).

FcRn affinity enhanced Fc variants to improve both antibody cytotoxicity and half-life were identified in screens at pH 6.0. The selected IgG variants can be produced as low fucosylated molecules. The resulting variants show increased serum persistence in hFcRn mice, as well as conserved enhanced ADCC (Monnet et al.) Exemplary variants include (with reference to IgG1 and Kabat numbering):
P230T/V303A/K322R/N389T/F404L/N434S; P228R/N434S;
Q311R/K334R/Q342E/N434Y; C226G/Q386R/N434Y; T307P/N389T/N434Y;
P230S/N434S; P230T/V305A/T307A/A378V/L398P/N434S; P23OT/P387S/N434S;
P230Q/E269D/N434S; N276S/A378V/N434S;
T307A/N315D/A330V/382V/N389T/N434Y; T256N/A378V/S383N/N434Y;
N315D/A330V/N361D/A387V/N434Y; V259I/N315D/M428L/N434Y;
P230S/N315D/M428L/N434Y; F241L/V264E/T307P/A378V/H433R;
T250A/N389K/N434Y; V305A/N315D/A330V/P395A/N434Y;
V264E/Q386R/P396L/N434S/K439R; E294del/T307P/N434Y (wherein ‘del’ indicates a deletion).

### Domain

The term “domain” refers to a folded polypeptide structure that retains its tertiary structure independent of the rest of the polypeptide. Generally, domains are responsible for discrete functional properties of polypeptides and in many cases may be added, removed or transferred to other polypeptides without loss of function of the remainder of the protein and/or of the domain.

### Single Variable Domain

The term “single variable domain” refers to a folded polypeptide domain comprising sequences characteristic of antibody variable domains. It, therefore, includes complete antibody variable domains such as VH, VHH and VL and modified antibody variable domains, for example, in which one or more loops have been replaced by sequences that are not characteristic of antibody variable domains, or antibody variable domains that have been truncated or comprise N- or C-terminal extensions, as well as folded fragments of variable domains that retain at least the binding activity and specificity of the full-length domain. A single variable domain as defined herein is capable of binding an antigen or epitope independently of a different variable region or domain. A “domain antibody” or “DAB” may be considered the same as a human “single variable domain”. A single variable domain may be a human single variable domain but also includes single variable domains from other species such as rodent (for example, as disclosed in WO 00/29004), nurse shark and Camelid VHHs Camelid VHHs are immunoglobulin single variable domain polypeptides that are derived from species including camel, llama, alpaca, dromedary, and guanaco, which produce heavy chain only antibodies naturally devoid of light chains. Such VHH domains may be humanized according to standard techniques available in the art, and such domains are considered to be “single variable domains”.

### Protein Scaffold

An antigen binding fragment may be provided by means of arrangement of one or more CDRs on non-antibody protein scaffolds. “Protein scaffold” as used herein includes but is not limited to an immunoglobulin (Ig) scaffold, for example, an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example, an IgG, or IgA scaffold. The IgG scaffold may comprise some or all the domains of an intact antibody (i.e., CH1, CH2, CH3, VH, VL). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human-crystallin and human ubiquitin (affilins); PDZ domains; scorpion toxin kunitz type domains of human protease inhibitors; and fibronectin/adnectin; which has been subjected to protein engineering in order to obtain binding to an antigen, such as the CD4 binding site of gp120.

### Attachment Inhibitor

As used herein, “attachment inhibitor” refers to a class of small molecule drugs that bind to the gp120 protein on the outer surface of HIV, preventing HIV from binding to and entering CD4+T lymphocytes (CD4 cells) and/or that bind the gp120 protein that is expressed on the outer surface of HIV infected CD4 cells. Examples of attachment inhibitor include temsavir and fostemsavir.

### Temsavir

As used herein, temsavir refers to 1-(4-benzoylpiperazin-1-yl)-2-[4-methoxy-7-(3-methyl-1,2,4-triazol-1-yl)-1H-pyrrolo[2,3-c]pyridin-3-yl]ethane-1,2-dione or compound (I), as disclosed in U.S. Pat. No. 7,354,924.

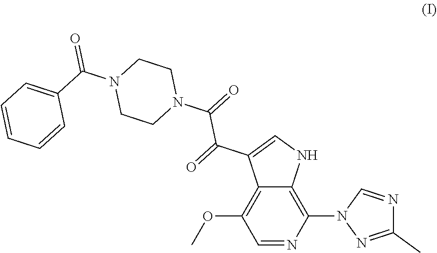 [SMILES: COc1cnc(-n2cnc(C)n2)c2[nH]cc(C(=O)C(=O)N3CCN(C(=O)c4ccccc4)CC3)c12]

### Fostemsavir

As used herein, fostemsavir refers to 1-benzoyl-4-[2-[4-methoxy-7-(3-methyl-1H-1,2,4-triazol-1yl)-1-[(phosphonooxy) methyl]-1H-pyrrolo[2,3-c]pyridin-3-yl]-1,2-dioxoethyl]-piperazine or compound (II), as disclosed in U.S. Pat. Nos. 7,745,625; 8,168,615; and 8,461,333.

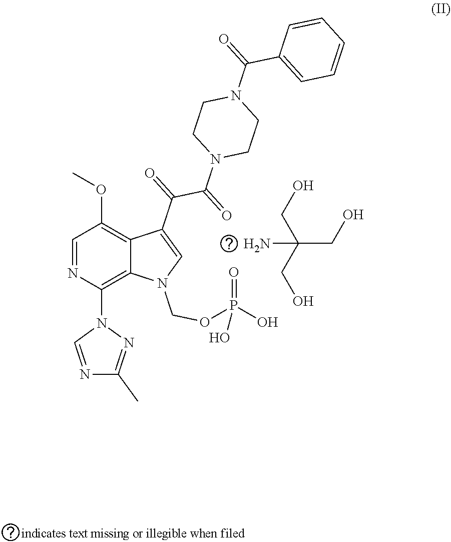

Fostemsavir is a prodrug that metabolizes to temsavir (methyl phosphate replaced by H). The un-phosphonated compound is 1-benzoyl-4-[2-[4-methoxy-7-(3-methyl-1H-1,2,4-triazol-1yl)-1H-pyrrolo[2,3-c]pyridin-3-yl]-1,2-dioxoethyl]-piperazine.

### Producer Cell

As used here, “producer cell” refers to a group of cells that are targeted and infected by HIV. This group of cells then facilitate HIV replication. CD4+T Lymphocytes and Macrophages fall under this category of producer cells.

### Prodrug

As used here, “prodrug” refers to a bio-reversible derivative of a drug molecule that undergoes an enzymatic and/or chemical transformation in vivo to release the active drug, which can then exert the desired pharmacological effect.

### Pharmaceutical Compositions

As used herein, the term “pharmaceutical composition” means a composition that is suitable for pharmaceutical use. Antigen binding proteins, antibodies, inhibitors, and integrase inhibitors as described herein may be incorporated into pharmaceutical compositions for use in the treatment of the human diseases described herein. In one embodiment, the pharmaceutical composition comprises an antigen binding protein in combination with one or more pharmaceutically acceptable carriers and/or excipients.

### Pharmaceutically Acceptable Salt

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011.

Suitable pharmaceutically acceptable salts can include acid or base addition salts. Suitable pharmaceutically acceptable salts of the invention include base addition salts.

Representative pharmaceutically acceptable base addition salts include, but are not limited to, aluminum, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

### Therapeutically Effective Amount

As used herein, the term “therapeutically effective amount” or “effective amount” refers to that amount of the compound being administered that will prevent a condition or will relieve to some extent one or more of the symptoms of the disorder being treated. Pharmaceutical compositions suitable for use herein include compositions wherein the active ingredients are contained in an amount sufficient enough to achieve the intended purpose. Determination of a therapeutically effective amount is well within the capability of those skilled in the art, especially in light of the detailed description provided herein.

### Treat

As used herein, the term “treatment,” “treating” or “treat” in the context of therapeutic methods, refers to alleviating the specified condition, eliminating or reducing the symptoms of the condition, slowing or eliminating the progression, invasion, or spread of the condition and reducing or delaying the reoccurrence of the condition in a previously afflicted human. The present invention further provides use of the compounds or compositions of the invention for the preparation of a medicament for the treatment of several conditions in a mammal (e.g., human) in need thereof.

### Prevent

As used herein, the term “prevention,” “preventing” or “prevent” in the context of therapeutic methods, refers to precluding the specified condition or symptoms of the condition, or in the occurrence of prior infection, precluding the re-occurrence of the condition. The present invention further provides use of the compounds of the invention for the preparation of a medicament for the prevention of several conditions in a mammal (e.g., human) in need thereof.

### Neutralize

As used herein, the term “neutralize” or “neutralizing,” refers to reducing the biological activity of HIV antigens, for example ENV or gp120, in the presence of an antigen binding protein in comparison to the antigen activity in the absence of the antigen binding protein, in vitro or in vivo. Neutralization of HIV may be due to preventing virus entry via interaction with its receptor.

### HIV Antigen Recognition

As used herein, the term “HIV antigen recognition,” “antigen recognition,” or “ENV detection”, refers to the binding of ENV on the infected cell surface by a CD4bs binding protein.

### Clearance

As used herein, the term “clearance,” “clear” or “clearing” refers to the killing of HIV infected cells. As an example, in one embodiment, “clearance” refers to a therapeutic administration or a combination of administrations that alone or in combination with one or more other compounds induces removal, elimination, or killing of cells that harbor replication competent HIV genome that can produce viruses. Clearance can be reflected as reduced levels of HIV DNA measured by e.g., a polymerase chain reaction (PCR) test. Clearance can also be reflected by delayed detection of HIV viruses when the therapeutic intervention is stopped.

### Cure

As used herein, the term “cure,” “curing” or “viral remission” in the context of therapeutic methods, refers to the eradication, stoppage, halt or end of the human immunodeficiency virus or symptoms, or the progression of the symptoms or virus, for a defined period. As an example, in one embodiment, “cure” or “curing” refers to a therapeutic administration or a combination of administrations that alone or in combination with one or more other compounds induces and maintains sustained viral control (undetectable levels of plasma viremia by, e.g., a polymerase chain reaction (PCR) test, a branched chain DNA (bDNA) test or a nucleic acid sequence based amplification (NASBA) test) of human immunodeficiency virus after a minimum of two years without any other therapeutic intervention. The above PCR, bDNA and NASBA tests are carried out using techniques known and familiar to one skilled in the art. As an example, the eradication, stoppage, halt or end of the human immunodeficiency virus or symptoms, or the progression of the symptoms or virus, may be sustained for a minimum of two years.

### Parenteral

As used herein, the term “parenteral” or “parenterally” in the context of therapeutic methods, refers to a route of administration of a pharmaceutical compound or composition other than by oral administration. Parenteral routes of administration suitable for use herein include injection, infusion, implantation or some other route other than the alimentary canal. Parenteral routes of injection administration include intravenous, intramuscular and subcutaneous.

### Closed Conformation

As used herein, the term “closed conformation” or “state 1 conformation” or “prefusion conformation” or “pre-triggered state” refers to a state of HIV ENV trimer organization prior to CD4 receptor engagement that exposes its heavily N-linked glycan covered outer surface that confers evasions of neutralizing antibody recognition. Interaction with the CD4 receptor causes ENV structure rearrangement and conformational transition into an intermediate open state, which leads to exposure of co-receptor binding sites and epitopes that are shielded in the closed conformation.

### STATEMENT OF THE INVENTION

### Methods of Treating HIV

The method of the present invention may be used to treat, clear, neutralize, prevent or cure Human Immunodeficiency Virus (HIV). In one aspect, the present invention provides a method of treating or preventing HIV comprising administering a therapeutically effective amount of two or more agents selected from the group consisting of: a CD4bs binding protein, a gp120 binding protein, a broadly neutralizing antibody or an antigen binding fragment thereof, at least one agent selected from the group consisting of: fostemsavir and temsavir, or a pharmaceutically acceptable salt thereof, and an integrase inhibitor or a pharmaceutically acceptable salt thereof.

In one aspect, there is provided a method for the treatment or prevention of HIV infections in a human in need thereof comprising administering a therapeutically effective amount of: (a) a first agent that comprises at least one agent selected from the group consisting of: fostemsavir and temsavir, or a pharmaceutically acceptable salt thereof; and (b) a second agent that comprises at least one broadly neutralizing antibody or an antigen binding fragment thereof.

In one aspect, there is provided a method for the treatment or prevention of HIV infections in a human in need thereof comprising administering a therapeutically effective amount of: (a) a first agent that comprises at least one agent selected from the group consisting of: fostemsavir and temsavir, or a pharmaceutically acceptable salt thereof; (b) a second agent that comprises at least one broadly neutralizing antibody or an antigen binding fragment thereof; and (c) a third agent comprising at least one integrase inhibitor or a pharmaceutically acceptable salt thereof.

In some embodiments, there is provided a method for the treatment or prevention of HIV infections in a human in need thereof comprising administering a therapeutically effective amount of: (a) a first agent that comprises at least one agent selected from the group consisting of: fostemsavir and temsavir, or a pharmaceutically acceptable salt thereof, and (b) a second agent that comprises at least one broadly neutralizing antibody or an antigen binding fragment thereof. In some embodiments, the first agent is fostemsavir or a pharmaceutically acceptable salt thereof. In some embodiments, the first agent is temsavir or a pharmaceutically acceptable salt thereof.

In some embodiments, the second agent is at least one broadly neutralizing antibody or an antigen binding fragment thereof that binds to at least one HIV glycoprotein selected from the group consisting of: HIV gp160, HIV gp120, and HIV gp41. In some embodiments, the at least one broadly neutralizing antibody binds to HIV gp160. In some embodiments, the at least one broadly neutralizing antibody binds to HIV gp120. In some embodiments, the at least one broadly neutralizing antibody binds to HIV gp41. In some embodiments, the second agent is at least one antibody selected from the group consisting of: 2G12, 2F5, 3BC176, 3BNC60, 3BNC1-17, 4E10, 8ANC131, 8ANC195, 10E8, 10-1074, 12A12, 35022, b12, B2530, CH01-04, CH103, CH31, HJ16, M66.6, N6, N6LS, N6-DE, N6-LAGA, NIH45-46, PG9, PG16, PGDM1400, PGT121, PGT128, PGT135, PGT141-PGT145, PGT151, PGV04, VRC01, VRC01-LS, VRC07, VRC07-523, VRC07-LS, and Z13. In some embodiments, the second agent is at least one antibody selected from the group consisting of: N6, N6LS, N6-DE, N6-LAGA, VRC01, VRC01-LS, VRC07, VRC07-523, and VRC07-LS. In some embodiments, the second agent is at least one antibody selected from the group consisting of: N6, N6LS, N6-DE, VRC01, VRC01-LS, VRC07, VRC07-523, and VRC07-LS. In some embodiments, the second agent is at least one antibody selected from the group consisting of: N6, N6LS, and N6 or N6LS having any one of ADCC mutations disclosed herein. In some embodiments, the second agent is an isolated monoclonal antibody or an antigen binding fragment thereof, comprising: a heavy chain complementarity determining region (CDRH) having a CDRH1 amino acid sequence that comprises a sequence that is at least 95%, 98%, 99% or 100% identical to SEQ ID NO: 1, a CDRH2 amino acid sequence that comprises a sequence that is at 95%, 98%, 99% or 100% identical to SEQ ID NO: 2, and a CDRH3 amino acid sequence that comprises a sequence that is at least 95%, 98%, 99% or 100% identical to SEQ ID NO: 3, and a light chain complementarity determining region (CDRL) having a CDRL1 amino acid that comprises a sequence that is at least 95%, 98%, 99% or 100% identical to SEQ ID NO: 4, a CDRL2 amino acid sequence that comprises a sequence that is at least 95%, 98%, 99% or 100% identical to SEQ ID NO: 5, and a CDRH3 amino acid sequence that comprises a sequence that is at least 95%, 98%, 99% or 100% identical to SEQ ID NO: 6. In some embodiment, the second agent is an isolated monoclonal antibody or an antigen binding fragment, comprising a heavy chain variable region (VH) having at least 95%, 98%, 99% or 100% sequence identity to SEQ ID NO: 7. In some embodiments, the second agent is an isolated monoclonal antibody or an antigen binding fragment, comprising a light chain variable region (VL) having at least 95%, 98%, 99% or 100% sequence identity to SEQ ID NO: 8. In some embodiments, the isolated monoclonal antibody further comprises a recombinant constant domain comprising M428L and N434S mutations. In some embodiments, the isolated monoclonal antibody further comprises a recombinant constant domain comprising S239D and 1332E mutations. In some embodiments, the isolated monoclonal antibody further comprises a recombinant constant domain comprising L235A and G237A mutations.

In some embodiments, the second agent is an isolated N6 monoclonal antibody or an antigen binding fragment thereof, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6. In some embodiments, the second agent is an isolated N6LS monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising M428L and N434S mutations. In some embodiments, the second agent is an isolated N6-DE monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising S239D and 1332E mutations. In some embodiments, the second agent is an isolated N6-LAGA monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising L235A and G237A mutations. In some embodiments, the antigen binding fragment is a Fv, Fab, F(ab′)₂, scFv or a scFV2 fragment.

In some embodiments, the method further comprises administering a therapeutically effective amount of a third agent comprising at least one integrase inhibitor or a pharmaceutically acceptable salt thereof. In some embodiments, the third agent is at least one agent selected from the group consisting of: raltegravir, elvitegravir, dolutegravir, bictegravir, and cabotegravir. In some embodiments, the third agent is raltegravir or cabotegravir. In some embodiments, the third agent is cabotegravir. In some embodiments, the second agent is N6 with at least one Fc modification in the constant domain as disclosed herein. In some embodiments, the second agent is N6LS. In some embodiments, the second agent is N6-DE. In some embodiments, the first agent is temsavir and the second agent is selected from the group consisting of N6LS and N6-DE, and the third agent is cabotegravir. In some embodiment, the first agent is temsavir and the second agent is N6LS, and the third agent is cabotegravir. In some embodiments, the first agent is temsavir and the second agent is N6-DE, and the third agent is cabotegravir. In some embodiments, the first agent is fostemsavir and the second agent is N6LS, and the third agent is cabotegravir. In some embodiments, the first agent is fostemsavir and the second agent is N6-DE, and the third agent is cabotegravir.

In some embodiments, the method according to any one of preceding embodiments, each of the first agent, the second agent, and the third agent is in the form of a pharmaceutical composition. In some embodiments, the first agent is administered prior to the administration of the second agent. In some embodiments, the method comprises administering about 1 mg/kg to 100 mg/kg body weight of the first agent to the human orally once a day, twice a day, or three times a day. In some embodiments, the method comprises administering about 1 mg/kg to 100 mg/kg body weight of the first agent to the human parenterally once a day, twice a day, or three times a day.

In some embodiments, the human is diagnosed with human immunodeficiency virus 1 (HIV-1) infection. In some embodiments, the human has previously been treated with one or more different HIV treatment modalities. In some embodiments, the human has been treated with therapeutic regimens of antiretroviral drug combinations termed, highly active antiretroviral therapy (“HAART”). In some embodiments, the human has been treated with modern antiretroviral therapy (ART).

In one aspect, the present invention provides a method of treating or preventing HIV in a human in need thereof, comprising administering to the human a therapeutically effective amount of: (a) a first agent comprising at least one agent selected from the group consisting of: fostemsavir and temsavir, or a pharmaceutically acceptable salt thereof, and (b) a second agent selected from the group consisting of: a CD4bs binding protein, a gp120 binding protein, and an integrase inhibitor or a pharmaceutically acceptable salt thereof.

In one aspect, the present invention provides a method of treating or preventing HIV in a human in need thereof, comprising administering to the human a therapeutically effective amount of: (a) a first agent comprising at least one agent selected from the group consisting of: fostemsavir and temsavir, or a pharmaceutically acceptable salt thereof, (b) a second agent comprising at least one CD4bs binding protein, and (c) a third agent comprising at least one integrase inhibitor or a pharmaceutically acceptable salt thereof.

According to one aspect, the present invention provides a method of treating HIV infection in a human in need thereof comprising administering a therapeutically effective amount of at least one agent selected from the group consisting of: fostemsavir and temsavir, or a pharmaceutically acceptable salt thereof to the human and sequentially administering a therapeutically effective amount of a CD4bs binding protein wherein the CD4bs binding protein is administered after the at least one agent selected from the group consisting of: fostemsavir and temsavir, or a pharmaceutically acceptable salt thereof.

According to another aspect, the present invention provides a method of clearing HIV infected cells from a human in need thereof comprising administering a therapeutically effective amount of at least one agent selected from the group consisting of: fostemsavir and temsavir, or a pharmaceutically acceptable salt thereof and a therapeutically effective amount of a CD4bs binding protein. In an embodiment of the method of clearing HIV infected cells, administering the at least one agent selected from the group consisting of: fostemsavir and temsavir, or a pharmaceutically acceptable salt thereof and the CD4bs binding protein is simultaneous. In an embodiment of the method of clearing HIV infected cells, the CD4bs binding protein is administered after the at least one agent selected from the group consisting of: fostemsavir and temsavir, or a pharmaceutically acceptable salt thereof.

According to another aspect, the present invention provides a method of preventing HIV infection in a human in need thereof comprising administering a therapeutically effective amount of at least one agent selected from the group consisting of: fostemsavir and temsavir, or a pharmaceutically acceptable salt thereof to the human and sequentially administering a therapeutically effective amount of a CD4 binding site (CD4bs) binding protein wherein CD4bs binding protein is administered after the at least one agent selected from the group consisting of: fostemsavir and temsavir, or a pharmaceutically acceptable salt thereof.

According to another aspect, the present invention provides a method of neutralizing HIV infection in a human in need thereof comprising administering a therapeutically effective amount of at least one agent selected from the group consisting of: fostemsavir and temsavir, or a pharmaceutically acceptable salt thereof to the human and sequentially administering a therapeutically effective amount of a CD4 binding site (CD4bs) binding protein wherein the CD4bs binding protein is administered after the administration of at least one agent selected from the group consisting of: fostemsavir and temsavir, or a pharmaceutically acceptable salt thereof.

According to another aspect, the present invention provides a method of curing HIV infection in a human in need thereof comprising administering a therapeutically effective amount of at least one agent selected from the group consisting of: fostemsavir and temsavir, or a pharmaceutically acceptable salt thereof to the human and sequentially administering a therapeutically effective amount of CD4 binding site (CD4bs) binding protein wherein the CD4bs binding protein is administered after the administration of at least one agent selected from the group consisting of: fostemsavir and temsavir, or a pharmaceutically acceptable salt thereof.

In some embodiments, the at least one agent is temsavir or fostemsavir. In an embodiment, the at least one agent is temsavir. In some embodiments, the at least one agent is fostemsavir. Without binding to any theory or mechanisms of action, temsavir or fostemsavir may work by blocking the gp120 receptor of the virus, which prevents initial viral attachment to the host CD4+ cell and entry into the host CD4+ cell. In some embodiments, administration of the at least one agent selected from the group consisting of: fostemsavir and temsavir, or a pharmaceutically acceptable salt thereof is oral or parenteral. In an embodiment of the methods, administration of the temsavir is parenteral. In some embodiments, administration of the fostemsavir is oral. In some embodiments, the at least one agent selected from the group consisting of: fostemsavir and temsavir, or a pharmaceutically acceptable salt thereof is administered in a dose range of 1 mg/kg to 100 mg/kg body weight. In some embodiments, the dose of the at least one agent selected from the group consisting of: fostemsavir and temsavir, or a pharmaceutically acceptable salt thereof is 100 mg to 1200 mg. In an embodiment of the methods, the dose of fostemsavir is 600 mg. In some embodiments, the administration of the at least one agent selected from the group consisting of: fostemsavir and temsavir, or a pharmaceutically acceptable salt thereof is a daily dose, a twice-daily dose, or a three times daily dose. In some embodiments, the administration is a twice-daily dose. In some embodiments, the administration is a daily dose. In an embodiment of the methods, the administration is a three times a day dose.

In some embodiments, the at least one agent selected from the group consisting of: fostemsavir and temsavir, or a pharmaceutically acceptable salt thereof locks gp120 onto the host cell membrane surface. In some embodiments, the at least one agent selected from the group consisting of: fostemsavir and temsavir, or a pharmaceutically acceptable salt thereof locks the gp120 into a closed conformation. In some embodiments, the at least one agent selected from the group consisting of: fostemsavir and temsavir, or a pharmaceutically acceptable salt thereof prevents cell membrane bound CD4 from interacting with the host cell expressed gp120. In some embodiments, the at least one agent selected from the group consisting of: fostemsavir and temsavir, or a pharmaceutically acceptable salt thereof enables or facilitates the binding of a CD4bs binding protein. In some embodiments, binding of the CD4bs binding protein to the HIV infected host cell is measured by flow cytometry using Mean Fluorescent Intensity (MFI).

In some embodiments, the methods comprise a gp120 binding protein. gp120 binding proteins bind to the gp120 found on the HIV virion or the infected host cell. In some embodiments, the gp120 binding protein comprises a CD4bs binding protein. In some embodiments, the CD4bs binding protein is at least one CD4bs binding protein. In some embodiments, the CD4bs binding protein is an antibody or binding fragment thereof. In some embodiments, the antibody is a broadly neutralizing antibody or binding fragment thereof. In some embodiments, the broadly neutralizing antibody is selected from the group consisting of N6, N6LS, N6-DE and N6-LAGA.

In some embodiments, the CD4bs binding protein comprises a heavy chain complementarity determining region (CDRH) having a CDRH1 amino acid sequence that comprises a sequence that is at least 80%, 85%, 90%, 95%, 98%, 99% or 100% identical to SEQ ID NO: 1, a CDRH2 amino acid sequence that comprises a sequence that is at least 80%, 85%, 90%, 95%, 98%, 99% or 100% identical to SEQ ID NO: 2, and a CDRH3 amino acid sequence that comprises a sequence that is at least 80%, 85%, 90%, 95%, 98%, 99% or 100% identical to SEQ ID NO: 3, and a light chain complementarity determining region (CDRL) having a CDRL1 amino acid that comprises a sequence that is at least 80%, 85%, 90%, 95%, 98%, 99% or 100% identical to SEQ ID NO: 4, a CDRL2 amino acid sequence that comprises a sequence that is at least 80%, 85%, 90%, 95%, 98%, 99% or 100% identical to SEQ ID NO: 5, and a CDRHL 3 amino acid sequence that comprises a sequence that is at least 80%, 85%, 90%, 95%, 98%, 99% or 100% identical to SEQ ID NO: 6.

In some embodiments, the CD4bs binding protein comprises a heavy chain variable region (VH) amino acid sequence that comprises a sequence that is at least 80%, 85%, 90%, 95%, 98%, 99% or 100% identical to a sequence selected from the group consisting of SEQ ID NO: 7. In some embodiments, the CD4bs binding protein comprises a light chain variable region (VL) amino acid sequence that comprises a sequence that is at least 80%, 85%, 90%, 95%, 98%, 99% or 100% identical to a sequence selected from the group consisting of SEQ ID NO: 8. In some embodiments, the CD4bs binding protein comprises a heavy chain (HC) amino acid sequence that comprises a sequence that is at least 80%, 85%, 90%, 95%, 98%, 99% or 100% identical to a sequence selected from the group consisting of SEQ ID NOS: 9, 11, 13 and 14. In some embodiments, the CD4bs binding protein comprises a light chain (LC) amino acid sequence that comprises a sequence that is at least 80%, 85%, 90%, 95%, 98%, 99% or 100% identical to a sequence selected from the group consisting of SEQ ID NO: 10 and 12.

In some embodiments, the CD4bs binding protein antibody comprises a full-length immunoglobulin CD4bs binding protein. In an embodiment of the methods, the full-length immunoglobulin CD4bs binding protein comprises a Fragment crystallizable region or “Fc” region or “constant region”. The Fc of any of the defined antibodies may include one or more amino acid substitutions to optimize in vivo half-life of the antibody. The serum half-life of IgG Abs is regulated by the neonatal Fc receptor (FcRn). In some embodiments, the antibody includes an amino acid substitution that increases binding to the FcRn. Several such substitutions are known to the person of ordinary skill in the art, such as substitutions at IgG constant regions T250Q and M428L (see, e.g., Hinton et al., J Immunol, 176:346-356, 2006); M428L and N434S (the “LS” mutation, see, e.g., Zalevsky, et al., Nature Biotechnology, 28:157-159, 2010); N434A (see, e.g., Petkova et al., Int. Immunol, 18:1759-1769, 2006); T307A, E380A, and N434A (see, e.g., Petkova et al., Int. Immunol, 18:1759-1769, 2006); and M252Y, S254T, and T256E (see, e.g., Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions. In some embodiments, the antibody comprises a recombinant constant domain comprising a modification that increases binding to a neonatal Fc receptor relative to an unmodified constant domain, wherein the recombinant domain is an IgG1 constant domain comprising M428L and N434S mutations.

In some embodiments, the constant region of any of the defined antibodies includes one or more amino acid substitutions to optimize antibody-dependent cell-mediated cytotoxicity (ADCC). ADCC is mediated primarily through a set of closely related Fc gamma receptors (FcγR). In some embodiments, the antibody includes one or more amino acid substitutions that increase binding to FcγRIIIa. Several such substitutions are known to the person of ordinary skill in the art, such as substitutions at IgG constant regions S239D and 1332E (see, e.g., Lazar et al., Proc. Natl, Acad. Sci. U.S.A., 103:4005-4010, 2006); S239D, A330L, and 1332E (see, e.g., Lazar et al., Proc. Natl, Acad. Sci. U.S.A., 103:4005-4010, 2006).

In some embodiments, combinations of the above substitutions are also included, to generate an IgG constant region with increased binding to FcRn and FcγRIIIa. The combinations increase antibody half-life and ADCC. For example, such combinations include antibodies with the following amino acid substitution in the Fc region: (1) S239D/1332E and T250Q/M428L; (2) S239D/1332E and M428L/N434S; (3) S239D/1332E and N434A; (4) S239D/1332E and T307A/E380A/N434A; (5) S239D/1332E and M252Y/S254T/T256E; (6) S239D/A330L/1332E and 250Q/M428L; (7) S239D/A330L/1332E and M428L/N434S; (8) S239D/A330L/1332E and N434A; (9) S239D/A330L/1332E and T307A/E380A/N434A; or (10) S239D/A330L/1332E and M252Y/S254T/T256E. In some embodiments, the antibodies, or an antigen binding fragment thereof is modified such that it is directly cytotoxic to infected cells, or uses natural defenses such as complement, antibody dependent cellular cytotoxicity (ADCC), or phagocytosis by macrophages.

In some embodiments, the antibody is an antibody fragment. In some embodiments, the antibody fragment is selected from a Fab′ fragment, a F (ab)′2 fragment, a single chain Fv protein (“scFv”), a disulfide stabilized Fv protein (“dsFv”), a diabody and a TANDABS. In an embodiment of the methods, the antibody fragment is scFv.

In some embodiments, the CD4bs binding protein comprises a recombinant constant domain comprising a modification that increases binding to a neonatal Fc receptor relative to an unmodified constant domain, wherein the recombinant domain is an IgG1 constant domain comprising M428L and N434S (“LS”) mutations.

In some embodiments, the CD4bs binding protein comprises a recombinant constant domain comprising a modification that affects antibody-dependent cell-mediated cytotoxicity (ADCC) relative to an unmodified constant domain, wherein the recombinant domain is an IgG1 constant domain comprising S239D/1332E (“DE”) mutation or L235A/G237A (“LAGA”) mutation.

In some embodiments, the CD4bs binding protein is selected from the group consisting of N6, N6LS, N6-LAGA and N6-DE. In some embodiments, the CD4bs binding protein is N6. In some embodiments, the CD4bs binding protein is N6LS. In some embodiments, the CD4bs binding protein is N6-LAGA. In some embodiments, the CD4bs binding protein is N6-DE.

In some embodiments, the CD4bs binding protein is sequentially administered at least 30 minutes, at least 1 hour, at least 2 hours, at least 4 hours or at least 6 hours after administering with the at least one agent selected from the group consisting of: fostemsavir and temsavir, or a pharmaceutically acceptable salt thereof. In some embodiments, the CD4bs binding protein is administered at least 30 minutes after administering with the at least one agent selected from the group consisting of: fostemsavir and temsavir, or a pharmaceutically acceptable salt thereof. In some embodiments, the CD4bs binding protein is administered at least 1 hour after administering with the at least one agent selected from the group consisting of: fostemsavir and temsavir, or a pharmaceutically acceptable salt thereof. In an embodiment of the methods, the CD4bs binding protein is administered at least 2 hours after administering with the at least one agent selected from the group consisting of: fostemsavir and temsavir, or a pharmaceutically acceptable salt thereof. In some embodiments, the CD4bs binding protein is administered at least 4 hours after administering with the at least one agent selected from the group consisting of: fostemsavir and temsavir, or a pharmaceutically acceptable salt thereof. In an embodiment of the methods, the CD4bs binding protein is administered at least 6 hours after administering with the at least one agent selected from the group consisting of: fostemsavir and temsavir, or a pharmaceutically acceptable salt thereof. In some embodiments, the CD4bs binding protein is administered at least 24 hours after administering with the at least one agent selected from the group consisting of: fostemsavir and temsavir, or a pharmaceutically acceptable salt thereof. In an embodiment of the methods, the CD4bs binding protein is administered at least 48 hours after administering with the at least one agent selected from the group consisting of: fostemsavir and temsavir, or a pharmaceutically acceptable salt thereof. In some embodiments, the CD4bs binding protein is administered at least 72 hours after administering with the at least one agent selected from the group consisting of: fostemsavir and temsavir, or a pharmaceutically acceptable salt thereof. In some embodiments, the CD4bs binding protein is administered at least 96 hours after administering with the at least one agent selected from the group consisting of: fostemsavir and temsavir, or a pharmaceutically acceptable salt thereof. Without being bound by theory, administering at least one agent selected from the group consisting of: fostemsavir and temsavir, or a pharmaceutically acceptable salt thereof to a human prior to the administration of a CD4 binding site (CD4bs) binding protein enhances CD4bs binding protein binding to HIV ENV. It is believed that this is because administering at least one agent selected from the group consisting of: fostemsavir and temsavir, or a pharmaceutically acceptable salt thereof to a human alters intracellular glycosylation and/or processing of the ENV trimer, promoting CD4bs binding protein engagement and HIV antigen recognition. It is also believed that administering at least one agent selected from the group consisting of: fostemsavir and temsavir, or a pharmaceutically acceptable salt thereof to a human locks or affixes the ENV trimer to the host cell surface membrane preventing ENV shedding and promoting bnAb engagement.

In some embodiments, the methods further comprise at least a second administration of the at least one agent selected from the group consisting of: fostemsavir and temsavir, or a pharmaceutically acceptable salt thereof and the CD4bs binding protein simultaneously after the sequential administration.

In various embodiments, the method comprises administering at least one integrase inhibitor. Integrase inhibitors, namely integrase strand transfer inhibitors (INSTIs), are actives which function by hindering integration of the retroviral. Exemplary compounds include, but are not limited to, raltegravir, elvitegravir, dolutegravir, bictegravir, and cabotegravir. In some embodiments, the at least one integrase inhibitor comprises cabotegravir.

### Combination for Use in Treatment of HIV

In one aspect, the present invention provides a pharmaceutical composition for use in treatment of HIV comprising two or more agents selected from the group consisting of: a CD4bs binding protein, a gp120 binding protein, at least one agent selected from the group consisting of: fostemsavir and temsavir, or a pharmaceutically acceptable salt thereof, and an integrase inhibitor or a pharmaceutically acceptable salt thereof.

In one aspect, the present invention provides a combination for use in the treatment or prevention of HIV, comprising administering to a human a first pharmaceutical composition comprising the first agent that comprises at least one agent selected from the group consisting of: fostemsavir and temsavir, or a pharmaceutically acceptable salt thereof with a second pharmaceutical composition comprising the second agent that comprises at least one broadly neutralizing antibody or an antigen binding fragment thereof. In some embodiments, the combination for use in the treatment or prevention of HIV further comprises administering a third pharmaceutical composition comprising the third agent comprising at least one integrase inhibitor or a pharmaceutically acceptable salt thereof.

In some embodiments, the first agent is temsavir or a pharmaceutically acceptable salt thereof. Yet in other embodiments, the first agent is fostemsavir or a pharmaceutically acceptable salt thereof.

In some embodiments, the second agent binds to HIV envelop glycoprotein or HIV gp120. In some embodiments, the second agent is at least one agent selected from the group consisting of: 2G12, 2F5, 3BC176, 3BNC60, 3BNC1-17, 4E10, 8ANC131, 8ANC195, 10E8, 10-1074, 12A12, 35022, b12, B2530, CH01-04, CH103, CH31, HJ16, M66.6, N6, N6LS, N6-DE, N6-LAGA, NIH45-46, PG9, PG16, PGDM1400, PGT121, PGT128, PGT135, PGT141-PGT145, PGT151, PGV04, VRC01, VRC01-LS, VRC07, VRC07-523, VRC07-LS, and Z13. In some embodiments, the second agent is at least one antibody selected from the group consisting of: N6, N6LS, N6-DE, N6-LAGA, VRC01, VRC01-LS, VRC07, VRC07-523, and VRC07-LS. In some embodiments, the second agent is at least one antibody selected from the group consisting of: N6, N6LS, N6-DE, VRC01, VRC01-LS, VRC07, VRC07-523, and VRC07-LS. In some embodiments, the second agent is at least one antibody selected from the group consisting of: N6, N6LS, and N6 or N6LS having any one of ADCC mutations disclosed herein. In some embodiments, the second agent is an isolated monoclonal antibody or an antigen binding fragment, comprising: a heavy chain complementarity determining region (CDRH) having a CDRH1 amino acid sequence that comprises a sequence that is at least 95%, 98%, 99% or 100% identical to SEQ ID NO: 1, a CDRH2 amino acid sequence that comprises a sequence that is at 95%, 98%, 99% or 100% identical to SEQ ID NO: 2, and a CDRH3 amino acid sequence that comprises a sequence that is at least 95%, 98%, 99% or 100% identical to SEQ ID NO: 3, and a light chain complementarity determining region (CDRL) having a CDRL1 amino acid that comprises a sequence that is at least 95%, 98%, 99% or 100% identical to SEQ ID NO: 4, a CDRL2 amino acid sequence that comprises a sequence that is at least 95%, 98%, 99% or 100% identical to SEQ ID NO: 5, and a CDRH3 amino acid sequence that comprises a sequence that is at least 95%, 98%, 99% or 100% identical to SEQ ID NO: 6. In some embodiment, the second agent is an isolated monoclonal antibody or an antigen binding fragment, comprising a heavy chain variable region (VH) having at least 95%, 98%, 99% or 100% sequence identity to SEQ ID NO: 7. In some embodiments, the second agent is an isolated monoclonal antibody or an antigen binding fragment, comprising a light chain variable region (VL) having at least 95%, 98%, 99% or 100% sequence identity to SEQ ID NO: 8. In some embodiments, the isolated monoclonal antibody further comprises a recombinant constant domain comprising M428L and N434S mutations. In some embodiments, the isolated monoclonal antibody further comprises a recombinant constant domain comprising S239D and 1332E mutations. In some embodiments, the isolated monoclonal antibody further comprises a recombinant constant domain comprising L235A and G237A mutations.

In some embodiments, the second agent is an isolated N6 monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2; and a CDRH3 amino acid sequence of SEQ ID NO: 3, and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6. In some embodiments, the second agent is an isolated N6LS monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2; a CDRH3 amino acid sequence of SEQ ID NO: 3, and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising M428L and N434S mutations. In some embodiments, the second agent is an isolated N6-DE monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2; a CDRH3 amino acid sequence of SEQ ID NO: 3, and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising S239D and 1332E mutations. In some embodiments, the second agent is an isolated N6-LAGA monoclonal antibody or an antigen binding fragment, comprising a heavy chain complementarity determining region (CDRH) having a CDRH1 amino acid sequence of SEQ ID NO: 1, a CDRH2 amino acid sequence of SEQ ID NO: 2; a CDRH3 amino acid sequence of SEQ ID NO: 3, and a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and a recombinant constant domain comprising L235A and G237A mutations. In some embodiments, the antigen binding fragment is a Fv, Fab, F(ab′)₂, scFv or a scFV2 fragment.

In some embodiments, the combination for use further comprises administering a therapeutically effective amount of a third pharmaceutical composition comprising a third agent comprising at least one integrase inhibitor or a pharmaceutically acceptable salt thereof. In some embodiments, the third agent is at least one agent selected from the group consisting of: raltegravir, elvitegravir, dolutegravir, bictegravir, and cabotegravir. In some embodiments, the third agent is raltegravir or cabotegravir. In some embodiments, the third agent is cabotegravir. In some embodiments, the first agent is temsavir and the second agent is selected from the group consisting of N6LS and N6-DE, and the third agent is cabotegravir. In some embodiments, the second agent is N6 with at least one ADCC mutation in the constant domain as disclosed herein. In some embodiments, the second agent is N6LS. In some embodiments, the second agent is N6-DE.

In some embodiments, the combination for use comprises administering the first pharmaceutical composition to the human prior to the administration of the second pharmaceutical composition. In some embodiments, about 1 mg/kg to 100 mg/kg body weight of the first pharmaceutical composition is administered to the human orally once a day, twice a day, or three times a day. In some embodiments, about 1 mg/kg to 100 mg/kg body weight of the first pharmaceutical composition is administered to the human parenterally once a day, twice a day, or three times a day.

In one aspect, the present invention provides a pharmaceutical composition for use in treatment of HIV comprising (a) a first agent comprising at least one agent selected from the group consisting of: fostemsavir and temsavir, or a pharmaceutically acceptable salt thereof, and (b) a second agent selected from the group consisting of: a CD4bs binding protein, a gp120 binding protein, and an integrase inhibitor or a pharmaceutically acceptable salt thereof.

In one aspect, the present invention provides a pharmaceutical composition for use in treatment of HIV comprising (a) a first agent comprising at least one agent selected from the group consisting of: fostemsavir and temsavir, or a pharmaceutically acceptable salt thereof, (b) a second agent comprising at least one CD4bs binding protein, and (c) a third agent comprising at least one integrase inhibitor or a pharmaceutically acceptable salt thereof.

According to another aspect, the present invention provides a combination for use in treatment of HIV comprising a therapeutically effective amount of at least one agent selected from the group consisting of: fostemsavir and temsavir, or a pharmaceutically acceptable salt thereof and a therapeutically effective amount of a CD4bs binding protein. In some embodiments, a human is administered the at least one agent selected from the group consisting of: fostemsavir and temsavir, or a pharmaceutically acceptable salt thereof and then sequentially administered the CD4bs binding protein for the use in treatment of HIV.

According to another aspect, the present invention provides a combination comprising a therapeutically effective amount of at least one agent selected from the group consisting of: fostemsavir and temsavir, or a pharmaceutically acceptable salt thereof and a therapeutically effective amount of a CD4bs binding protein, wherein a human is administered the at least one agent selected from the group consisting of: fostemsavir and temsavir, or a pharmaceutically acceptable salt thereof and the CD4bs binding protein for the use in clearing HIV infected cells. In an embodiment of the combination for the use of clearing HIV infected cells, administering the at least one agent selected from the group consisting of: fostemsavir and temsavir, or a pharmaceutically acceptable salt thereof and the CD4bs binding protein is simultaneous. In an embodiment of the combination for the use of clearing HIV infected cells, the CD4bs binding protein is administered after the administration of at least one agent selected from the group consisting of: fostemsavir and temsavir, or a pharmaceutically acceptable salt thereof. According to another aspect, the present invention provides a combination comprising a therapeutically effective amount of at least one agent selected from the group consisting of: fostemsavir and temsavir, or a pharmaceutically acceptable salt thereof and a CD4bs binding protein wherein a human is administered the at least one agent selected from the group consisting of: fostemsavir and temsavir, or a pharmaceutically acceptable salt thereof and then sequentially administered the CD4bs binding protein for the use in prevention of HIV.

According to another aspect, the present invention provides a combination comprising a therapeutically effective amount of at least one agent selected from the group consisting of: fostemsavir and temsavir, or a pharmaceutically acceptable salt thereof and a CD4bs binding protein wherein a human is administered the at least one agent selected from the group consisting of: fostemsavir and temsavir, or a pharmaceutically acceptable salt thereof and then sequentially administered the CD4bs binding protein for the use in neutralization of HIV.

According to another aspect, the present invention provides a combination comprising a therapeutically effective amount of at least one agent selected from the group consisting of: fostemsavir and temsavir, or a pharmaceutically acceptable salt thereof and a CD4bs binding protein wherein a human is administered the at least one agent selected from the group consisting of: fostemsavir and temsavir, or a pharmaceutically acceptable salt thereof and then sequentially administered the CD4bs binding protein for the use in cure of HIV.

In some embodiments, the at least one agent is temsavir or a pharmaceutically acceptable salt thereof. In an embodiment of the combinations for use, the at least one agent is fostemsavir or a pharmaceutically acceptable salt thereof.

In some embodiments, administration of the at least one agent selected from the group consisting of: fostemsavir and temsavir, or a pharmaceutically acceptable salt thereof is oral or parenteral. In some embodiments, administration of the temsavir is parenteral. In some embodiments, administration of the fostemsavir is oral.

In some embodiments, the at least one agent selected from the group consisting of: fostemsavir and temsavir, or a pharmaceutically acceptable salt thereof is administered in a dose range of 1 mg/kg to 100 mg/kg body weight. In some embodiments, the dose of the at least one agent selected from the group consisting of: fostemsavir and temsavir, or a pharmaceutically acceptable salt thereof is 100 mg to 1000 mg. In some embodiments, the dose of fostemsavir 600 mg.

In some embodiments, the administration of the at least one agent selected from the group consisting of: fostemsavir and temsavir, or a pharmaceutically acceptable salt thereof is a daily dose, a twice-daily dose or a three times daily dose. In some embodiments, the administration is a twice-daily dose. In some embodiments, the administration is a daily dose. In some embodiments, the administration is a three times daily dose.

In some embodiments, the at least one agent selected from the group consisting of: fostemsavir and temsavir, or a pharmaceutically acceptable salt thereof locks gp120 onto the host cell membrane surface. In some embodiments, the at least one agent selected from the group consisting of: fostemsavir and temsavir, or a pharmaceutically acceptable salt thereof locks the gp120 into a closed conformation. In an embodiment of the combinations for use, the at least one agent selected from the group consisting of: fostemsavir and temsavir, or a pharmaceutically acceptable salt thereof prevents cell membrane-bound CD4 from interacting with the host cell expressed gp120. In some embodiments, the at least one agent selected from the group consisting of: fostemsavir and temsavir, or a pharmaceutically acceptable salt thereof enables or facilitates the binding of the CD4bs binding protein. In some embodiments, binding of the CD4bs binding protein to the HIV infected host cell is measured by flow cytometry using Mean Fluorescent Intensity (MFI).

In some embodiments, the combinations for use comprise a gp120 binding protein. gp120 binding proteins bind to the gp120 found on the HIV virion or the infected host cell. In some embodiments, the gp120 binding protein comprises a CD4bs binding protein. In some embodiments, the CD4bs binding protein is at least one CD4bs binding protein. In some embodiments, the CD4bs binding protein is an antibody or binding fragment thereof. In some embodiments, the antibody is a broadly neutralizing antibody or binding fragment thereof. In some embodiments, the broadly neutralizing antibody is selected from the group consisting of N6, N6LS, N6-DE and N6-LAGA.

In some embodiments, the CD4bs binding protein comprises a heavy chain complementarity determining region (CDRH) having a CDRH1 amino acid sequence that comprises a sequence that is at least 80%, 85%, 90%, 95%, 98%, 99% or 100% identical to SEQ ID NO: 1, a CDRH2 amino acid sequence that comprises a sequence that is at least 80%, 85%, 90%, 95%, 98%, 99% or 100% identical to SEQ ID NO: 2, and a CDRH3 amino acid sequence that comprises a sequence that is at least 80%, 85%, 90%, 95%, 98%, 99% or 100% identical to SEQ ID NO: 3, and a light chain complementarity determining region (CDRL) having a CDRL1 amino acid that comprises a sequence that is at least 80%, 85%, 90%, 95%, 98%, 99% or 100% identical to SEQ ID NO: 4, a CDRL2 amino acid sequence that comprises a sequence that is at least 80%, 85%, 90%, 95%, 98%, 99% or 100% identical to SEQ ID NO: 5, and a CDRHL 3 amino acid sequence that comprises a sequence that is at least 80%, 85%, 90%, 95%, 98%, 99% or 100% identical to SEQ ID NO: 6.

In some embodiments, the CD4bs binding protein comprises a heavy chain variable region (VH) amino acid sequence that comprises a sequence that is at least 80%, 85%, 90%, 95%, 98%, 99% or 100% identical to a sequence selected from the group consisting of SEQ ID NO: 7.

In some embodiments, the CD4bs binding protein comprises a light chain variable region (VL) amino acid sequence that comprises a sequence that is at least 80%, 85%, 90%, 95%, 98%, 99% or 100% identical to a sequence selected from the group consisting of SEQ ID NO: 8.

In some embodiments, the CD4bs binding protein comprises a heavy chain (HC) amino acid sequence that comprises a sequence that is at least 80%, 85%, 90%, 95%, 98%, 99% or 100% identical to a sequence selected from the group consisting of SEQ ID NOS: 9, 11, 13 and 14.

In some embodiments, the CD4bs binding protein comprises a light chain (LC) amino acid sequence that comprises a sequence that is at least 80%, 85%, 90%, 95%, 98%, 99% or 100% identical to a sequence selected from the group consisting of SEQ ID NO: 10 and 12.

In some embodiments, the CD4bs binding protein antibody comprises a full-length immunoglobulin CD4bs binding protein. In some embodiments, the full-length immunoglobulin CD4bs binding protein comprises a Fragment crystallizable region or “Fc” region or “constant region”. The Fc of any of the defined antibodies may include one or more amino acid substitutions to optimize in vivo half-life of the antibody. The serum half-life of IgG Abs is regulated by the neonatal Fc receptor (FcRn). In an embodiment of the combinations for use, the antibody includes an amino acid substitution that increases binding to the FcRn. Several such substitutions are known to the person of ordinary skill in the art, such as substitutions at IgG constant regions T250Q and M428L (see, e.g., Hinton et al., J Immunol, 176:346-356, 2006); M428L and N434S (the “LS” mutation, see, e.g., Zalevsky, et al., Nature Biotechnology, 28:157-159, 2010); N434A (see, e.g., Petkova et al., Int. Immunol, 18:1759-1769, 2006); T307A, E380A, and N434A (see, e.g., Petkova et al., Int. Immunol, 18:1759-1769, 2006); and M252Y, S254T, and T256E (see, e.g., Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions. In an embodiment of the combinations for use, the antibody comprises a recombinant constant domain comprising a modification that increases binding to a neonatal Fc receptor relative to an unmodified constant domain, wherein the recombinant domain is an IgG1 constant domain comprising M428L and N434S mutations.

In some embodiments, the constant region of any of the defined antibodies includes one or more amino acid substitutions to optimize ADCC. ADCC is mediated primarily through a set of closely related Fcy receptors. In an embodiment of the combinations for use, the antibody includes one or more amino acid substitutions that increase binding to FcγRIIIa. Several such substitutions are known to the person of ordinary skill in the art, such as substitutions at IgG constant regions S239D and 1332E (see, e.g., Lazar et al., Proc. Natl, Acad. Sci. U.S.A., 103:4005-4010, 2006); S239D, A330L, and 1332E (see, e.g., Lazar et al., Proc. Natl, Acad. Sci. U.S.A., 103:4005-4010, 2006).

In some embodiments, combinations of the above substitutions are also included, to generate an IgG constant region with increased binding to FcRn and FcγRIIIa. The combinations increase antibody half-life and ADCC. For example, such combinations include antibodies with the following amino acid substitution in the Fc region: (1) S239D/1332E and T250Q/M428L; (2) S239D/1332E and M428L/N434S; (3) S239D/1332E and N434A; (4) S239D/1332E and T307A/E380A/N434A; (5) S239D/1332E and M252Y/S254T/T256E; (6) S239D/A330L/1332E and 250Q/M428L; (7) S239D/A330L/1332E and M428L/N434S; (8) S239D/A330L/1332E and N434A; (9) S239D/A330L/1332E and T307A/E380A/N434A; or (10) S239D/A330L/1332E and M252Y/S254T/T256E. In some examples, the antibodies, or an antigen binding fragment thereof is modified such that it is directly cytotoxic to infected cells, or uses natural defenses such as complement, ADCC, or phagocytosis by macrophages.

In some embodiments, the antibody is an antibody fragment. In an embodiment of the combinations for use, the antibody fragment is selected from a Fab′ fragment, a F (ab)′2 fragment, a single chain Fv protein (“scFv”), a disulfide stabilized Fv protein (“dsFv”), a diabody and a TANDABS. In an embodiment of the combinations for use, the antibody fragment is scFv.

In some embodiments, the CD4bs binding protein comprises a recombinant constant domain comprising a modification that increases binding to a neonatal Fc receptor relative to an unmodified constant domain, wherein the recombinant domain is an IgG1 constant domain comprising M428L and N434S (“LS”) mutations.

In some embodiments, the CD4bs binding protein comprises a recombinant constant domain comprising a modification that affects ADCC relative to an unmodified constant domain, wherein the recombinant domain is an IgG1 constant domain comprising S239D/1332E (“DE”) mutation.

In some embodiments, the CD4bs binding protein is selected from the group consisting of N6, N6LS, N6-LAGA and N6-DE. In some embodiments, the CD4bs binding protein is N6. In some embodiments, the CD4bs binding protein is N6LS. In some embodiments, the CD4bs binding protein is N6-LAGA. In some embodiments, the CD4bs binding protein is N6-DE.

In some embodiments, the CD4bs binding protein is sequentially administered selected from a group consisting of at least 30 minutes, at least 1 hour, at least 2 hours, at least 4 hours and at least 6 hours after administering with the at least one agent selected from the group consisting of: fostemsavir and temsavir, or a pharmaceutically acceptable salt thereof. In some embodiments, the CD4bs binding protein is administered at least 30 minutes after administering with the at least one agent selected from the group consisting of: fostemsavir and temsavir, or a pharmaceutically acceptable salt thereof. In an embodiment of the combinations for use, the CD4bs binding protein is administered at least 1 hour after administering with the at least one agent selected from the group consisting of: fostemsavir and temsavir, or a pharmaceutically acceptable salt thereof. In some embodiments, the CD4bs binding protein is administered at least 2 hours after administering with the at least one agent selected from the group consisting of: fostemsavir and temsavir, or a pharmaceutically acceptable salt thereof. In an embodiment of the combinations for use, the CD4bs binding protein is administered at least 4 hours after administering with the at least one agent selected from the group consisting of: fostemsavir and temsavir, or a pharmaceutically acceptable salt thereof. In some embodiments, the CD4bs binding protein is administered at least 6 hours after administering with the at least one agent selected from the group consisting of: fostemsavir and temsavir, or a pharmaceutically acceptable salt thereof. In some embodiments, the CD4bs binding protein is administered at least 24 hours after administering with the at least one agent selected from the group consisting of: fostemsavir and temsavir, or a pharmaceutically acceptable salt thereof. In some embodiments, the CD4bs binding protein is administered at least 48 hours after administering with the at least one agent selected from the group consisting of: fostemsavir and temsavir, or a pharmaceutically acceptable salt thereof. In an embodiment of the combinations for use, the CD4bs binding protein is administered at least 72 hours after administering with the at least one agent selected from the group consisting of: fostemsavir and temsavir, or a pharmaceutically acceptable salt thereof. In some embodiments, the CD4bs binding protein is administered at least 96 hours after administering with the at least one agent selected from the group consisting of: fostemsavir and temsavir, or a pharmaceutically acceptable salt thereof.

In some embodiments, the combination for use further comprises at least a second administering of the at least one agent selected from the group consisting of: fostemsavir and temsavir, or a pharmaceutically acceptable salt thereof and the CD4bs binding protein simultaneously after the sequential administering.

In various embodiments, the combinations of the present invention may also encompass integrase inhibitors. Integrase inhibitors, namely integrase strand transfer inhibitors (INSTIs), are actives which function by hindering integration of the retroviral. Exemplary compounds include, but are not limited to, raltegravir, elvitegravir, dolutegravir, bictegravir, and cabotegravir.

### Other Aspects

According to another aspect, the invention provides use of a therapeutically effective amount of at least one agent selected from the group consisting of: fostemsavir and temsavir, or a pharmaceutically acceptable salt thereof and a therapeutically effective amount of a CD4bs binding protein, as previously defined, in the manufacture of a medicament for use in the treatment of HIV, wherein a human is administered the at least one agent selected from the group consisting of: fostemsavir and temsavir, or a pharmaceutically acceptable salt thereof and then sequentially administered the CD4bs binding protein for the use in treatment of HIV.

According to another aspect, the invention provides use of a therapeutically effective amount of at least one agent selected from the group consisting of: fostemsavir and temsavir, or a pharmaceutically acceptable salt thereof and a therapeutically effective amount of a CD4bs binding protein, as previously defined, in the manufacture of a medicament for use in the treatment of HIV, wherein a human is administered the at least one agent selected from the group consisting of: fostemsavir and temsavir, or a pharmaceutically acceptable salt thereof and then sequentially administered the CD4bs binding protein for the use in prevention of HIV.

According to another aspect, the invention provides use of a therapeutically effective amount of at least one agent selected from the group consisting of: fostemsavir and temsavir, or a pharmaceutically acceptable salt thereof and a therapeutically effective amount of a CD4bs binding protein, as previously defined, in the manufacture of a medicament for use in the treatment of HIV, wherein a human is administered the at least one agent selected from the group consisting of: fostemsavir and temsavir, or a pharmaceutically acceptable salt thereof and then sequentially administered the CD4bs binding protein for the use in neutralization of HIV.

According to another aspect, the invention provides use of a therapeutically effective amount of at least one agent selected from the group consisting of: fostemsavir and temsavir, or a pharmaceutically acceptable salt thereof and a therapeutically effective amount of a CD4bs binding protein as previously defined in the manufacture of a medicament for use in the treatment of HIV, wherein a human is administered the at least one agent selected from the group consisting of: fostemsavir and temsavir, or a pharmaceutically acceptable salt thereof and then sequentially administered the CD4bs binding protein for the use in cure of HIV.

According to another aspect, the invention provides use of a therapeutically effective amount of at least one agent selected from the group consisting of: fostemsavir and temsavir, or a pharmaceutically acceptable salt thereof and therapeutically effective amount of a CD4bs binding protein as previously defined in the manufacture of a medicament for use in the clearance of HIV infected cells.

According to another aspect, the invention provides a kit comprising a therapeutically effective amount of at least one agent selected from the group consisting of: fostemsavir and temsavir, or a pharmaceutically acceptable salt thereof and a therapeutically effective amount of a CD4bs binding protein.

In some embodiments, the invention provides a method of treating HIV infection in a human in need thereof comprising administering a therapeutically effective amount of at least one agent selected from the group consisting of: fostemsavir and temsavir, or a pharmaceutically acceptable salt thereof to the human and a therapeutically effective amount of a CD4 binding site (CD4bs) binding protein wherein the CD4bs binding protein is administered after the administration of the at least one agent selected from the group consisting of: fostemsavir and temsavir, or a pharmaceutically acceptable salt thereof.

In some embodiments, the invention provides a method of clearing HIV infected cells from a human in need thereof, comprising administering a therapeutically effective amount of at least one agent selected from the group consisting of: fostemsavir and temsavir, or a pharmaceutically acceptable salt thereof and a therapeutically effective amount of a CD4bs binding protein.

In some embodiments, the invention provides a combination comprising a therapeutically effective amount of at least one agent selected from the group consisting of: fostemsavir and temsavir, or a pharmaceutically acceptable salt thereof and a therapeutically effective amount of a CD4bs binding protein, wherein a human is administered the at least one agent selected from the group consisting of: fostemsavir and temsavir, or a pharmaceutically acceptable salt thereof and then sequentially administered the CD4bs binding protein for the use in treatment of HIV. In some embodiment, a combination comprising a therapeutically effective amount of at least one agent selected from the group consisting of: fostemsavir and temsavir, or a pharmaceutically acceptable salt thereof and a therapeutically effective amount of a CD4bs binding protein, wherein the at least one agent selected from the group consisting of: fostemsavir and temsavir, or a pharmaceutically acceptable salt thereof and the CD4bs binding protein is administered to a human for the use in clearing HIV infected cells.

In some embodiments, the at least one agent selected from the group consisting of: fostemsavir and temsavir, or a pharmaceutically acceptable salt thereof and the CD4bs binding protein are administered concurrently, simultaneously, separately, or sequentially. In some embodiment, the at least one agent selected from the group consisting of: fostemsavir and temsavir, or a pharmaceutically acceptable salt thereof and the CD4bs binding protein are sequentially, wherein the CD4bs binding protein is administered after the administration of the at least one agent selected from the group consisting of: fostemsavir and temsavir, or a pharmaceutically acceptable salt thereof. In some embodiments, the at least one agent is temsavir or a pharmaceutically acceptable salt thereof. In other embodiments, the at least one agent is fostemsavir or a pharmaceutically acceptable salt thereof.

In some embodiments, the at least one agent selected from the group consisting of: fostemsavir and temsavir, or a pharmaceutically acceptable salt thereof is administered orally or parenterally. In some embodiments, the administration of temsavir is parenteral. In other embodiments, the administration of the fostemsavir is oral.

In some embodiments, the at least one agent selected from the group consisting of: fostemsavir and temsavir, or a pharmaceutically acceptable salt thereof is administered in a dose range of 1 mg/kg to 100 mg/kg body weight. In some embodiments, the at least one agent selected from the group consisting of: fostemsavir and temsavir, or a pharmaceutically acceptable salt thereof dose is 100 mg to 1200 mg. In some embodiments, the at least one agent selected from the group consisting of: fostemsavir and temsavir, or a pharmaceutically acceptable salt thereof is administered once a day, twice a day or three times a day. In one embodiment, the at least one agent selected from the group consisting of: fostemsavir and temsavir, or a pharmaceutically acceptable salt thereof is administered twice a day.

In some embodiments, the at least one agent selected from the group consisting of: fostemsavir and temsavir, or a pharmaceutically acceptable salt thereof facilitates the binding of the CD4bs binding protein. In some embodiments, the at least one agent selected from the group consisting of: fostemsavir and temsavir, or a pharmaceutically acceptable salt thereof locks a gp120 Envelope glycoprotein (gp120 ENV) in a closed conformation.

In some embodiments, the CD4bs binding protein comprises an antibody or binding fragment thereof that binds to the CD4bs. In some embodiments, the antibody is a broadly neutralizing antibody or binding fragment thereof. In some embodiments, the CD4bs binding protein comprises a heavy chain complementarity determining region (CDRH) having a CDRH1 amino acid sequence that comprises a sequence that is at least 80%, 85%, 90%, 95%, 98%, 99% or 100% identical to SEQ ID NO: 1, a CDRH2 amino acid sequence that comprises a sequence that is at least 80%, 85%, 90%, 95%, 98%, 99% or 100% identical to SEQ ID NO: 2, and a CDRH3 amino acid sequence that comprises a sequence that is at least 80%, 85%, 90%, 95%, 98%, 99% or 100% identical to SEQ ID NO: 3, and a light chain complementarity determining region (CDRL) having a CDRL1 amino acid that comprises a sequence that is at least 80%, 85%, 90%, 95%, 98%, 99% or 100% identical to SEQ ID NO: 4, a CDRL2 amino acid sequence that comprises a sequence that is at least 80%, 85%, 90%, 95%, 98%, 99% or 100% identical to SEQ ID NO: 5, and a CDRL3 amino acid sequence that comprises a sequence that is at least 80%, 85%, 90%, 95%, 98%, 99% or 100% identical to SEQ ID NO: 6.

In some embodiments, the CD4bs binding protein comprises a heavy chain variable region (VH) amino acid sequence that comprises a sequence that is at least 80%, 85%, 90%, 95%, 98%, 99% or 100% identical to SEQ ID NO: 7. In some embodiments, the CD4bs binding protein comprises a light chain variable region (VL) amino acid sequence that comprises a sequence that is at least 80%, 85%, 90%, 95%, 98%, 99% or 100% identical to SEQ ID NO: 8.

In some embodiments, the CD4bs binding protein comprises a heavy chain (HC) amino acid sequence that comprises a sequence that is at least 80%, 85%, 90%, 95%, 98%, 99% or 100% identical to a sequence selected from the group consisting of SEQ ID NOs: 9, 11, 13 and 14. In some embodiments, the CD4bs binding protein comprises a light chain (LC) amino acid sequence that comprises a sequence that is at least 80%, 85%, 90%, 95%, 98%, 99% or 100% identical to a sequence selected from the group consisting of SEQ ID NOs: 10 and 12.

In some embodiment, the CD4bs binding protein comprises a full-length immunoglobulin binding protein. In some embodiments, the CD4bs binding protein is a binding protein antibody fragment. In some embodiments, the CD4bs binding protein antibody fragment is a Fab′ fragment, a F (ab)′2 fragment, a single chain Fv protein (“scFv”), a disulfide stabilized Fv protein (“dsFv”), a diabody or a TANDABS. In some embodiments, the CD4bs binding protein antibody fragment is a scFv.

In some embodiments, the CD4bs binding protein comprises a recombinant constant domain comprising a modification that increases binding to a neonatal Fc receptor relative to an unmodified constant domain, wherein the recombinant domain is an IgG1 constant domain comprising M428L and N434S mutations. In some embodiments, the CD4bs binding protein comprises a recombinant constant domain comprising a modification that affects antibody-dependent cell-mediated cytotoxicity (ADCC) relative to an unmodified constant domain, wherein the recombinant domain is an IgG1 constant domain comprising a S239D/1332E mutation or a L235A/G237A mutation. In some embodiments, the CD4bs binding protein is a monoclonal antibody selected from the group consisting of N6, N6LS, N6-DE and N6-LAGA. In some embodiments, the CD4bs binding protein is N6. In some embodiments, the CD4bs binding protein is N6LS.

In some embodiments, the CD4bs binding protein is administered at least 30 minutes, at least 1 hour, at least 2 hours, at least 4 hours and at least 6 hours after administering the at least one agent selected from the group consisting of: fostemsavir and temsavir, or a pharmaceutically acceptable salt thereof. In some embodiments, the method further comprises administering at least one additional agent selected from the group consisting of: fostemsavir and temsavir, or a pharmaceutically acceptable salt thereof. In some embodiments, the method or the combination further comprises a third agent comprising at least one integrase inhibitor or a pharmaceutically acceptable salt thereof.

In some embodiments, use of a therapeutically effective amount of at least one agent selected from the group consisting of: fostemsavir and temsavir, or a pharmaceutically acceptable salt thereof and therapeutically effective amount of a CD4bs binding protein in the manufacture of a medicament for use in the treatment of HIV is provided. In some embodiments, the at least one agent selected from the group consisting of: fostemsavir and temsavir, or a pharmaceutically acceptable salt thereof and the CD4bs binding protein is administered to a human concurrently, simultaneously, separately, or sequentially. In some embodiments, use of a therapeutically effective amount of at least one agent selected from the group consisting of: fostemsavir and temsavir, or a pharmaceutically acceptable salt thereof and therapeutically effective amount of a CD4bs binding protein according to any of the embodiments disclosed herein is provided in the manufacture of a medicament for use in the clearance of HIV infected cells.

In one aspect, the present invention provides a kit comprising the first pharmaceutical composition comprising the first agent that comprises at least one agent selected from the group consisting of: fostemsavir and temsavir, or a pharmaceutically acceptable salt thereof, the second pharmaceutical composition comprising the second agent that comprises at least one broadly neutralizing antibody or an antigen binding fragment thereof, and optionally the third pharmaceutical composition comprising the third agent comprising at least one integrase inhibitor or a pharmaceutically acceptable salt thereof. In some embodiments, the invention provides a kit comprising a therapeutically effective amount of at least one agent selected from the group consisting of: fostemsavir and temsavir, or a pharmaceutically acceptable salt thereof and therapeutically effective amount of a CD4bs binding protein.

### EXAMPLES

### Example 1: Anti-Viral Activity

### SUMMARY

The effect of temsavir on the binding of the bnAbs N6, N6-DE and N6-LAGA to cell surface expressed HIV-1 envelope from a productive infection of human CD4⁺ T cells with the HIV-1 lab strain, HxB2, was assessed by flow cytometry.

### Procedure

Preparation and HIV-1 infection of target cells: Peripheral blood mononuclear cells (PBMCs) were obtained by continuous-flow leukapheresis from healthy donors, isolated by ficoll-hypaque density gradient centrifugation and cryopreserved in the vapor phase of liquid nitrogen. PBMCs were rapidly thawed to room temperature in a 37° C. water bath and cultured in assay medium (RPMI 1640 media supplemented with 10% fetal calf serum plus 30 U/mL recombinant IL-2) containing 2 ug/mL phytohemagglutinin-P (Sigma) for three days in a humidified incubator at 37° C., 5% CO₂. CD4⁺ T cells were isolated by negative selection (Stemcell Technologies), washed twice and resuspended with assay medium at a final density of 1-2×10⁶ cells/mL and incubated at 37° C., 5% CO₂ overnight. Cells were centrifuged at 500×g for 10 minutes, resuspended with assay medium at 5×10⁶ cell/mL and 200 uL (1×10⁶ total cells) were distributed to 24-well tissue culture treated plates. An HIV-1 lab strain, HxB2 virus, previously propagated in human PBMC cells, was diluted with assay medium containing 50 ug/mL DEAE-dextran and 200 uL was added to the cells in the 24-well plates. The plates were centrifuged at 1,200×g for 1.5 hours and the infected cells were collected and pooled in a 50 ml conical tube. The infected cells were washed twice and resuspended in assay medium to a final density of 1-2×10⁶ cells/mL and incubated at 37° C., 5% CO₂ for three days.

Treatment of HIV-infected target cells with temsavir and N6 bnAbs: HIV infected cells were centrifuged at 500×g for 10 minutes, resuspended with assay medium at 3×10⁶ cell/mL and 50 uL (150,000 total cells) were distributed to 96-well, U-bottom tissue-culture treated plates. Infected cell culture was then treated with temsavir that was dissolved in DMSO at a final concentration of 0.5 uM or DMSO only, as vehicle control, and the assay plates were incubated at 37° C., 5% CO₂ for 16-24 hours N6 bnAbs (N6, N6-DE and N6-LAGA) were produced by ChemPartner from HEK293 cells according to the sequences listed. The antibodies were eluted and diluted in buffer consisting of 150 mM Arginine, 50 mM Sodium acetate, pH 5.5, 150 mM NaCl) were serially titrated in assay medium and added to the temsavir-treated cells for a final concentration range of 50 ug/mL to 0.01 ug/mL. The plates were incubated at 37° C., 5% CO₂ for 30 minutes.

Flow cytometry: The assay plates were removed from the incubator and the cells were pelleted by centrifugation at 500×g for 2 minutes at 25° C. The cells were washed twice by mixing with 200 μL of ice cold wash buffer (phosphate buffered saline containing 2% fetal calf serum) with centrifugation at 500×g for 2 minutes at 25° C. for each wash cycle. The cells were resuspended with 100 uL ice cold wash buffer containing 1:2,000 LIVE/DEAD™ fixable aqua dead cell stain (Invitrogen), 1:400 BV786 mouse anti-human CD4 mAb (clone OKT4, BD Biosciences) and 1:200 R-Phycoerythrin F(ab′)₂ fragment goat anti-human IgG (Jackson Immuno Research) and incubated for 30 minutes at 4° C. in the dark. The stained cells were washed twice with wash buffer as indicated above, fixed and permeabilized with BD cytofix/cytoPerm™ before intracellular staining for 30 minutes at 4° C. with 50 uL FITC-anti-HIV-1 core antigen (KC57, Beckman Coulter) prepared at 1:200 in BD Perm/Wash™ buffer. The stained cells were washed twice with wash buffer as indicated above, acquired on a Fortessa cytometer (BD Biosciences) and the data were analyzed by mean fluorescent intensity (MFI) using FlowJo v10.8 software (Tree Star).

### Results

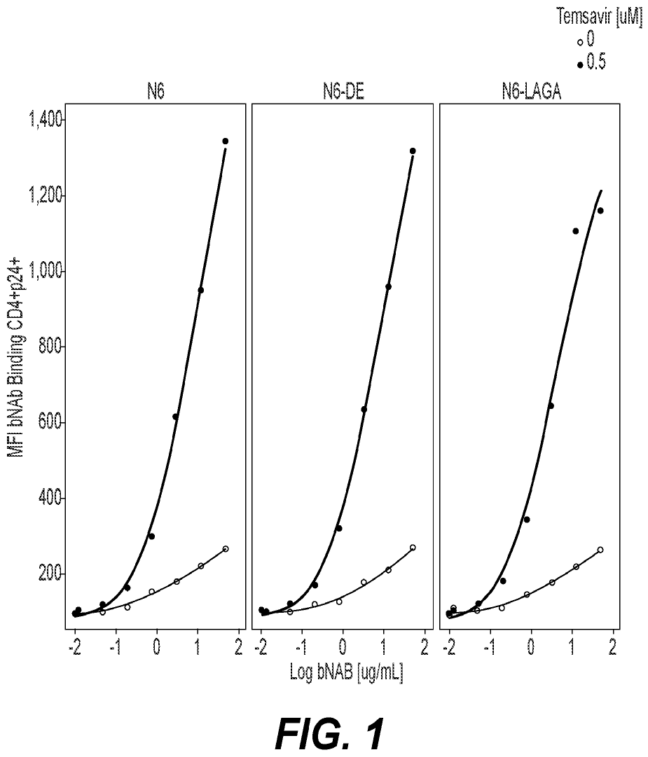

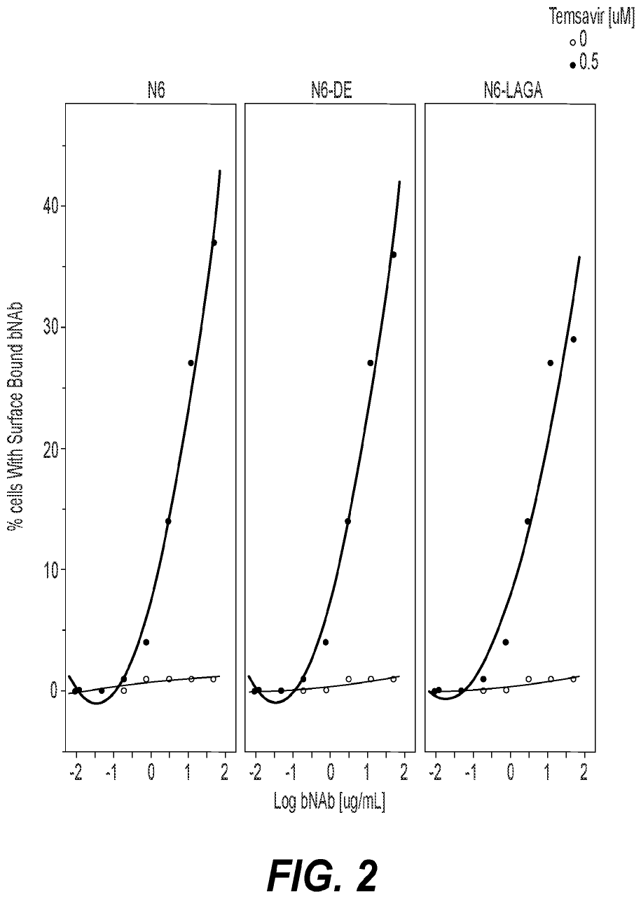

Treating HIV infected cells with 0.5 uM temsavir for 24 hr resulted in a substantial increase in the binding of the bnAbs N6, N6-DE and N6-LAGA to the cell surface of CD4⁺/p24⁺ cells (FIG. 1) compared to DMSO only treated cells. There was also a significant increase in the overall percentage of cells that contained bound bnAbs when treated with 0.5 uM temsavir compared to DMSO only treated cells (FIG. 2).

### Example 2: Antibody-Dependent Cellular Cytotoxicity

### Summary

Temsavir enhancement of killing of HIV infected cells mediated by three CD4 binding site bnAbs, N6, N6-DE and N6-LAGA, was assessed in an ADCC assay.

### Procedure

Isolation and preparation of target and effector cells: Peripheral blood mononuclear cells (PBMCs) were obtained by continuous-flow leukapheresis from healthy donors, isolated by ficoll-hypaque density gradient centrifugation and cryopreserved in the vapor phase of liquid nitrogen. PBMCs were rapidly thawed to room temperature in a 37° C. water bath and resuspended in RPMI 1640 media supplemented with 10% fetal calf serum. For the target cells, CD4+T lymphocytes were purified using immunomagnetic negative selection beads as per the manufacturer's instructions (StemCell Technologies). The cells were activated with phytohemagglutinin-P (Sigma; PHA-P; 5 μg/mL) for 72 hours at 37° C., 5% CO₂ and then maintained under the same incubation conditions in RPMI 1640 medium supplemented with 10% fetal calf serum plus 50 U/mL recombinant IL-2. Natural Killer (NK) effector cells were purified from autologous PBMCs using immunomagnetic negative selection beads as per the manufacturer's instructions (StemCell Technologies). The cells were maintained in RPMI 1640 medium supplemented with 10% fetal calf serum and incubated at 37° C., 5% CO₂ for 24 hr prior to ADCC assay.

Infection of target cells: HIV-1 lab strain IIIB was used to infect the activated CD4+T lymphocytes by spin infection at 1200×g for 1.5 hours in 24-well plates at 25° C. in the presence of 25 μg/mL DEAE-Dextran. The infected cells were collected, washed and resuspended in RPMI 1640 medium supplemented with 10% fetal calf serum plus 50 U/mL recombinant IL-2 and incubated at 37° C., 5% CO₂ for 3 days until infection rates reached above 20%.

ADCC assay: HIV infected CD4⁺ T cells (targets) were incubated with cell proliferation dye as per the manufacturer's instructions (eFluor670; eBioscience) and plated in 96-well U-bottom plates. Temsavir was dissolved in DMSO and diluted into RPMI 1640 medium supplemented with 10% fetal calf and serially titrated in the same medium in 5-fold increments for a dose range of 50 nM-0.08 nM. In order to exclude the possibility that reduction in percent of infected cells was a result of antiviral effect of N6 and temsavir, Raltegravir was added to the culture medium to prevent viral spread, which allows for analysis of infected cell elimination independent of blockade in viral infection effect of temsavir and N6. The temsavir titration was added to the dye-loaded target cells and the assay plates were incubated at 37° C., 5% CO₂ for 16-24 hours Antibodies were added to appropriate wells of the assay plates at a final concentration of 5 ug/mL and incubated at room temperature for 15 minutes. NK effector cells purified according to the Isolation and preparation of target and effector cells, previously mentioned, were added at an effector-to-target ratio of 3:1 and the assay plates were incubated at 37° C., 5% CO₂ for 24 hours.

Flow cytometry: Following the ADCC portion of the assay, cells were incubated for 20-30 minutes at room temperature with viability dye (LiveDeadAqua; ThermoFisher), anti-CD3-BV421 (clone SP34-2; BD Biosciences, 1:200 final concentration) and anti-CD4-BV786 (clone OKT4; BD Bioscience, 1:400 final concentration). The cells were then stained intracellularly for HIV-1 p24, using the Cytofix/Cytoperm Fixation/Permeabilization Kit (BD Biosciences) with subsequent addition of anti-p24 mAb (FITC anti-p24, clone KC57; Beckman Coulter/Immunotech; 1:200 final concentration) for 20-30 minutes at room temperature. Cells were resuspended in Pharmingen Stain Buffer (BSA) (BD Biosciences) containing 5×10⁴ flow cytometry particles/mL (AccuCount blank particles; 5.3 μm; Spherotech). Samples were acquired on a Fortessa cytometer (BD Biosciences) and data analysis was performed using FlowJo v10.8 software (Tree Star). The percentage of specific killing was calculated with the following formula: (% of p24⁺ cells in Targets plus Effectors)−(% of p24⁺ cells in Targets plus Effectors plus Abs)/(% of p24⁺ cells in Targets alone without antibody or temsavir) by gating on live target cells.

### Results

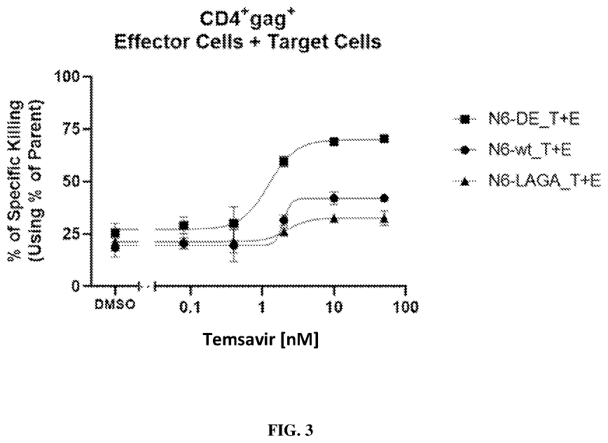

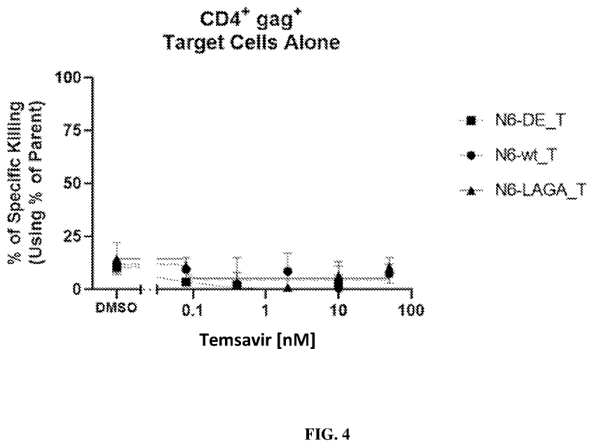

Temsavir enhances the NK-directed killing of HIV infected cells mediated by CD4 binding site bnAbs (FIG. 3). The Fc binding-enhanced version, N6-DE, facilitated the greatest magnitude of killing (75% maximum) whereas the Fc binding-reduced version, N6-LAGA, showed the least amount of killing (25% maximum), indicating a direct involvement of FcγRIB receptor function on NK-mediated killing of target cells. There was no evidence of killing when effector cells were eliminated from the assay (FIG. 4)

### Sequence Listings

| SEQ ID NO: | DESCRIPTION | SEQUENCE |
|---|---|---|
| 1 | N6 HCDR1 | AHILF |
| 2 | N6 HCDR2 | WIKPQYGAVNFGGGFRD |
| 3 | N6 HCDR3 | DRSYGDSSWALDA |
| 4 | N6 LCDR1 | QTSQGVGSDLH |
| 5 | N6 LCDR2 | HTSSVED |
| 6 | N6 LCDR3 | QVLQF |
| 7 | N6 VH | RAHLVQSGTAMKKPGASVRVSCQTSGYTFTA |
| | | HILFWFRQAPGRGLEWVGWIKPQYGAVNFGG |
| | | GFRDRVTLTRDVYREIAYMDIRGLKPDDTAVY |
| | | YCARDRSYGDSSWALDAWGQGTTVVVSA |
| 8 | N6 VL | YIHVTQSPSSLSVSIGDRVTINCQTSQGVGSDL |
| | | HWYQHKPGRAPKLLIHHTSSVEDGVPSRESGS |
| | | GFHTSFNLTISDLQADDIATYYCQVLQFFGRGS |
| | | RLHIK |
| 9 | N6 HC | RAHLVQSGTAMKKPGASVRVSCQTSGYTFTA |
| | | HILFWFRQAPGRGLEWVGWIKPQYGAVNFGG |
| | | GFRDRVTLTRDVYREIAYMDIRGLKPDDTAVY |
| | | YCARDRSYGDSSWALDAWGQGTTVVVSAAS |
| | | TKGPSVFPLAPSSKSTSGGTAALGCLVKDYFPE |
| | | PVTVSWNSGALTSGVHTFPAVLQSSGLYSLSS |
| | | VVTVPSSSLGTQTYICNVNHKPSNTKVDKKVE |
| | | PKSCDKTHTCPPCPAPELLGGPSVFLFPPKPKD |
| | | TLMISRTPEVTCVVVDVSHEDPEVKFNWYVD |
| | | GVEVHNAKTKPREEQYNSTYRVVSVLTVLHQ |
| | | DWLNGKEYKCKVSNKALPAPIEKTISKAKGQP |
| | | REPQVYTLPPSRDELTKNQVSLTCLVKGFYPS |
| | | DIAVEWESNGQPENNYKTTPPVLDSDGSFFLY |
| | | SKLTVDKSRWQQGNVFSCSVMHEALHNHYT |
| | | QKSLSLSPGK |
| 10 | N6 LC | YIHVTQSPSSLSVSIGDRVTINCQTSQGVGSDL |
| | | HWYQHKPGRAPKLLIHHTSSVEDGVPSRFSGS |
| | | GFHTSFNLTISDLQADDIATYYCQVLQFFGRGS |
| | | RLHIKRTVAAPSVFIFPPSDEQLKSGTASVVCL |
| | | LNNFYPREAKVQWKVDNALQSGNSQESVTEQ |
| | | DSKDSTYSLSSTLTLSKADYEKHKVYACEVTH |
| | | QGLSSPVTKSFNRGEC |
| 11 | N6LS HC | MGWSCIILFLVATATGVHSRAHLVQSGTAMK |
| | | KPGASVRVSCQTSGYTFTAHILFWFRQAPGRG |
| | | LEWVGWIKPQYGAVNFGGGFRDRVTLTRDVY |
| | | REIAYMDIRGLKPDDTAVYYCARDRSYGDSS |
| | | WALDAWGQGTTVVVSAASTKGPSVFPLAPSS |
| | | KSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDKTHTCPPC |
| | | PAPELLGGPSVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALPAPIEKTISKAKGQPREPQVYTLPPSR |
| | | DELTKNQVSLTCLVKGFYPSDIAVEWESNGQP |
| | | ENNYKTTPPVLDSDGSFFLYSKLTVDKSRWQQ |
| | | GNVFSCSVLHEALHSHYTQKSLSLSPGK |
| 12 | N6LS LC | MGWSCIILFLVATATGVHSYIHVTQSPSSLSVSI |
| | | GDRVTINCQTSQGVGSDLHWYQHKPGRAPKL |
| | | LIHHTSSVEDGVPSRFSGSGFHTSFNLTISDLQA |
| | | DDIATYYCQVLQFFGRGSRLHIKRTVAAPSVFI |
| | | FPPSDEQLKSGTASVVCLLNNFYPREAKVQWK |
| | | VDNALQSGNSQESVTEQDSKDSTYSLSSTLTLS |
| | | KADYEKHKVYACEVTHQGLSSPVTKSFNRGE |
| | | C |
| 13 | N6-LAGA | MGWSCIILFLVATATGVHSRAHLVQSGTAMK |
| | HC | KPGASVRVSCQTSGYTFTAHILFWFRQAPGRG |
| | | LEWVGWIKPQYGAVNFGGGFRDRVTLTRDVY |
| | | REIAYMDIRGLKPDDTAVYYCARDRSYGDSS |
| | | WALDAWGQGTTVVVSAASTKGPSVFPLAPSS |
| | | KSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDKTHTCPPC |
| | | PAPELAGAPSVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALPAPIEKTISKAKGQPREPQVYTLPPSR |
| | | DELTKNQVSLTCLVKGFYPSDIAVEWESNGQP |
| | | ENNYKTTPPVLDSDGSFFLYSKLTVDKSRWQQ |
| | | GNVFSCSVMHEALHNHYTQKSLSLSPGK |
| 14 | N6-DE HC | MGWSCIILFLVATATGVHSRAHLVQSGTAMK |
| | | KPGASVRVSCQTSGYTFTAHILFWFRQAPGRG |
| | | LEWVGWIKPQYGAVNFGGGFRDRVTLTRDVY |
| | | REIAYMDIRGLKPDDTAVYYCARDRSYGDSS |
| | | WALDAWGQGTTVVVSAASTKGPSVFPLAPSS |
| | | KSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDKTHTCPPC |
| | | PAPELLGGPDVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALPAPEEKTISKAKGQPREPQVYTLPPSR |
| | | DELTKNQVSLTCLVKGFYPSDIAVEWESNGQP |
| | | ENNYKTTPPVLDSDGSFFLYSKLTVDKSRWQQ |
| | | GNVFSCSVMHEALHNHYTQKSLSLSPGK |

1. A method for the treatment of Human Immunodeficiency Virus (HIV) infections in a human in need thereof comprising administering a therapeutically effective amount of:
(a) a first agent that comprises at least one agent selected from the group consisting of: fostemsavir and temsavir, or a pharmaceutically acceptable salt thereof, and
(b) a second agent that comprises at least one broadly neutralizing antibody or an antigen binding fragment thereof.

2. The method of claim 1, wherein the first agent is fostemsavir or a pharmaceutically acceptable salt thereof.

3. The method of claim 1, wherein the first agent is temsavir or a pharmaceutically acceptable salt thereof.

4. The method of claim 1, wherein the second agent binds to at least one HIV envelope glycoprotein selected from the group consisting of: HIV gp160, HIV gp120, and HIV gp41.

5. The method of claim 1, wherein the second agent binds to HIV gp120.

6. The method of claim 1, wherein the second agent is at least one agent selected from the group consisting of: 2G12, 2F5, 3BC176, 3BNC60, 3BNC1-17, 4E10, 8ANC131, 8ANC195, 10E8, 10-1074, 12A12, 35022, b12, B2530, CH01-04, CH103, CH31, HJ16, M66.6, N6, N6LS, N6-DE, N6-LAGA, NIH45-46, PG9, PG16, PGDM1400, PGT121, PGT128, PGT135, PGT141-PGT145, PGT151, PGV04, VRC01, VRC01-LS, VRC07, VRC07-523, VRC07-LS, and Z13.

7. The method of claim 1, wherein the second agent is an isolated monoclonal antibody or an antigen binding fragment thereof, comprising:
a heavy chain complementarity determining region (CDRH) having a CDRH1 amino acid sequence that comprises a sequence that is at least 95%, 98%, 99% or 100% identical to SEQ ID NO: 1, a CDRH2 amino acid sequence that comprises a sequence that is at 95%, 98%, 99% or 100% identical to SEQ ID NO: 2, and a CDRH3 amino acid sequence that comprises a sequence that is at least 95%, 98%, 99% or 100% identical to SEQ ID NO: 3, and
a light chain complementarity determining region (CDRL) having a CDRL1 amino acid that comprises a sequence that is at least 95%, 98%, 99% or 100% identical to SEQ ID NO: 4, a CDRL2 amino acid sequence that comprises a sequence that is at least 95%, 98%, 99% or 100% identical to SEQ ID NO: 5, and a CDRH3 amino acid sequence that comprises a sequence that is at least 95%, 98%, 99% or 100% identical to SEQ ID NO: 6.

8. The method of claim 1, wherein the second agent is an isolated monoclonal antibody or an antigen binding fragment thereof, comprising a heavy chain variable region (V_{H}) having at least 95%, 98%, 99% or 100% sequence identity to SEQ ID NO: 7.

9. The method of claim 1, wherein the second agent is an isolated monoclonal antibody or an antigen binding fragment thereof, comprising a light chain variable region (V_{L}) having at least 95%, 98%, 99% or 100% sequence identity to SEQ ID NO: 8.

10. The method of claim 7, wherein the isolated monoclonal antibody further comprises a recombinant constant domain comprising M428L and N434S mutations.

11. The method of claim 7, wherein the isolated monoclonal antibody further comprises a recombinant constant domain comprising S239D and 1332E mutations.

12. The method of claim 7, wherein the isolated monoclonal antibody further comprises a recombinant constant domain comprising L235A and G237A mutations.

13. The method of claim 1, wherein the second agent is an isolated monoclonal antibody (N6) or an antigen binding fragment thereof, comprising
a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3; and
a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6.

14. The method of claim 1, wherein the second agent is an isolated monoclonal antibody (N6LS) or an antigen binding fragment thereof, comprising
a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3;
a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and
a recombinant constant domain comprising M428L and N434S mutations.

15. The method of claim 1, wherein the second agent is an isolated monoclonal antibody (N6-DE) or an antigen binding fragment thereof, comprising
a heavy chain complementarity determining region (CDRH) having a CDRH1 amino acid sequence of SEQ ID NO: 1, a CDRH2 amino acid sequence of SEQ ID NO: 2, and a CDRH3 amino acid sequence of SEQ ID NO: 3;
a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and
a recombinant constant domain comprising S239D and 1332E mutations.

16. The method of claim 1, wherein the second agent is an isolated monoclonal antibody (N6-LAGA) or an antigen binding fragment thereof, comprising
a heavy chain complementarity determining region (CDRH) having a CDRH1 amino acid sequence of SEQ ID NO: 1, a CDRH2 amino acid sequence of SEQ ID NO: 2, a CDRH3 amino acid sequence of SEQ ID NO: 3;
a light chain complementarity determining region (CDRL) having a CDRL1 amino acid of SEQ ID NO: 4, a CDRL2 amino acid sequence of SEQ ID NO: 5, and a CDRH3 amino acid sequence of SEQ ID NO: 6; and
a recombinant constant domain comprising L235A and G237A mutations.

17. The method of claim 7, wherein the antigen binding fragment is a Fv, Fab, F(ab′)₂, scFv or a scFV₂ fragment.

18. The method of claim 1, further comprising administering a therapeutically effective amount of a third agent comprising at least one integrase inhibitor or a pharmaceutically acceptable salt thereof.

19. The method of claim 18, wherein the third agent comprises at least one agent selected from the group consisting of: raltegravir, elvitegravir, dolutegravir, bictegravir, and cabotegravir.

20. The method of claim 18, wherein the third agent is raltegravir or cabotegravir.

21. The method of claim 18, wherein the third agent is cabotegravir.

22. The method of claim 18, wherein the first agent is temsavir and the second agent is N6LS, and the third agent is cabotegravir.

23. The method of claim 18, wherein the first agent is temsavir and the second agent is N6-DE, and the third agent is cabotegravir.

24. The method of claim 18, wherein each of the first agent, the second agent, and the third agent is in the form of a pharmaceutical composition.

25. The method of claim 1, wherein the first agent is administered prior to the administration of the second agent.

26. The method of claim 1, wherein the method comprises administering about 1 mg/kg to 100 mg/kg body weight of the first agent to the human orally once a day, twice a day, or three times a day.

27. The method of claim 1, wherein the method comprises administering about 1 mg/kg to 100 mg/kg body weight of the first agent to the human parenterally once a day, twice a day, or three times a day.

28. The method of claim 1, wherein the human is diagnosed with human immunodeficiency virus 1 (HIV-1) infection.

29. The method of claim 1, wherein the human has previously been treated with one or more different HIV treatment modalities.

30-34. (canceled)

35. A kit comprising a first pharmaceutical composition comprising a first agent that comprises at least one agent selected from the group consisting of: fostemsavir and temsavir, or a pharmaceutically acceptable salt thereof, a second pharmaceutical composition comprising a second agent that comprises at least one broadly neutralizing antibody or an antigen binding fragment thereof, and optionally a third pharmaceutical composition comprising a third agent that comprises at least one integrase inhibitor or a pharmaceutically acceptable salt thereof.


SEQUENCE LISTING (14 sequences)

SEQ ID NO: 1 - protein, 5 aa, synthetic construct
AHILF

SEQ ID NO: 2 - protein, 17 aa, synthetic construct
WIKPQYGAVNFGGGFRD

SEQ ID NO: 3 - protein, 13 aa, synthetic construct
DRSYGDSSWALDA

SEQ ID NO: 4 - protein, 11 aa, synthetic construct
QTSQGVGSDLH

SEQ ID NO: 5 - protein, 7 aa, synthetic construct
HTSSVED

SEQ ID NO: 6 - protein, 5 aa, synthetic construct
QVLQF

SEQ ID NO: 7 - protein, 122 aa, synthetic construct [sequence omitted]

SEQ ID NO: 8 - protein, 103 aa, synthetic construct [sequence omitted]

SEQ ID NO: 9 - protein, 452 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 210 aa, synthetic construct
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 471 aa, synthetic construct
MGWSCIILFLVATATGVHSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 12 - protein, 229 aa, synthetic construct
MGWSCIILFLVATATGVHSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 471 aa, synthetic construct
MGWSCIILFLVATATGVHSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 471 aa, synthetic construct
MGWSCIILFLVATATGVHSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK